(19) United States
(12) Patent Application Publication
(10) Pub. No.: US 2005/0136079 A1
(43) Pub. Date: Jun. 23, 2005

(54) Cosmetic compositions containing fullerene clusters

(76) Inventors: Nail Burangulov, St. Petersburg (RU); Alexander P. Moravsky, Tucson, AZ (US); Yuiya V. Kulikova, St. Petersburg (RU); George I. Dyachuk, St. Petersburg (RU); Raouf O. Loutfy, Tucson, AZ (US)

Correspondence Address: SCULLY SCOTT MURPHY & PRESSER, PC, 400 GARDEN CITY PLAZA, SUITE 300, GARDEN CITY, NY, 11530, US

(21) Appl. No.: 10/858,361

(22) Filed: Jun. 1, 2004

Related U.S. Application Data
(60) Provisional application No. 60/475,203, filed on May 30, 2003.

(51) Int. Cl. A61K 7/021; A61K 33/44

(52) U.S. Cl. 424/401; 424/125; 424/63

(57) ABSTRACT

The present invention is directed to a cosmetic composition comprising effective amounts of fullerene clusters in association with cosmetically acceptable carrier to prevent or retard free radical oxidation processes in the skin and to the method of using said composition for reducing damage to healthy skin or mucous membranes by free radicals.

### CROSS REFERENCE TO RELATED APPLICATIONS

This application is claiming priority under 35 U.S.C. §l 19(e) of U.S. Provisional Patent Application No. 60/475,203 filed on May 30, 2003.

### FIELD OF THE INVENTION

This invention relates to ultrafine dispersed fullerene particles and their use in cosmetics.

### BACKGROUND OF THE INVENTION

A fullerene is a third allotrope of carbon which is different from other forms of carbon, such as diamond and graphite. It consists solely of carbon atoms arranged in a hollow closed cage. More specifically, a fullerene is comprised of closed polyhedra in which the carbon atoms are so arranged that they are linked together to form hexagons and pentagons. Stable fullerenes used in the present invention are produced in industrial or laboratory scale amounts. Examples include C₆₀, C₇₀, C₇₆, C₇₈, C₈₀, C₈₂, C₈₄ and higher molecular weight molecules.

Fullerenes were first described in an article by Kroto et al., in Nature, vol. 318, p. 163 (1985), in which their existence was postulated based upon mass-spectra of the products of laser vaporization of graphite. Fullerene, as a substance, was made available in 1990, when Huffman and Kratschmer found a method to prepare and isolate fullerenes, such as C₆₀ and C₇₀ in macroscopic amounts. See Kratschmer et al., Nature, vol. 347, 354 (1990).

The geometry and electronic structures of fullerenes impart to them unique physical properties and chemical reactivities. For example, the electron affinity of fullerenes is high. For example, C₆₀ has an electron affinity of 2.65 eV. They possess enhanced reactivity towards the addition of free radicals to carbon-carbon double bonds and towards nucleophilic attack by lone pair electrons. Addition of free radicals to fullerene double bonds causes formation of rather inactive fullerene-centered radicals that are unable to participate in free radical chain propagation reactions, for example, in free radical oxidation of organic compounds. Thus fullerenes are antioxidants. It has been stated that one fullerene molecule is capable of inactivating some tens of “hot” free radicals by sacrificing double bonds. Furthermore, the fullerene molecules can catalytically scavenge and recombine free radicals, due to the occurrence of reductive elimination of the attached organic groups from the fullerene cage in the form of appropriate dimers. This property imparts to a fullerene molecule a capability to convert free radicals into inactive molecular products.

The unique properties of fullerenes create peculiar physical and chemical features of fullerene solids. For example, the fullerene molecular crystal has an exceedingly high energy of intermolecular interaction, much greater than that of molecular crystals of the majority of similar size organic substances. This is attributable to the extended conjugated electronic structure of the fullerene cage, and the dense mode of fullerene molecules packing in the lattice of molecular crystals. This phenomenon is seen in crystals of pure C₆₀ and C₇₀ fullerenes, higher order-fullerenes, their mixtures, crystalline solvates of fullerenes with aromatic molecules, fullerenes adsorbed on graphite, etc.

The high strength of the fullerene molecular crystals results in their drastically different chemical and biochemical behavior compared to that of separate fullerene molecules, such as those in true solutions and prevents the fullerene molecule per se from being used in cosmetic, pharmaceutical or veterinary compositions. For example, the macroscopic size of a fullerene crystal does not allow its incorporation in skin tissues and other biological structures. Only the surface molecules, accounting for about 0.4% of all molecules in a common micrometer-size fullerene particle, are exposed to solution and thus allowed to react with solution components. Moreover, the specific reactivity of surface fullerene molecules is generally much lower than that of individual dissolved molecules.

There are only a limited number of solvents capable of dissolving fullerenes. Aromatic solvents and halogenated hydrocarbons, traditionally used for dissolving fullerenes, are strong toxins, thereby precluding their use in cosmetology and pharmaceutical and veterinary compositions.

Designers of cosmetics and medicinal drugs are trying to take advantage of various fullerene properties to overcome the difficulties of using fullerenes outlined hereinabove. The use of crystalline fullerenes in oil-in-water or water-in-oil emulsions, suspensions, powders, tinted creams, nail varnishes and other cosmetic compositions was described in U.S. Pat. No. 5,612,021 to Melull “Cosmetic Make-Up Composition Containing Fullerene or Mixture of Fullerenes as a Pigmenting Agent”. Fullerenes are used in these make-up compositions exclusively as a pigment or filler to allegedly endow these products with a certain color, as well as other useful cosmetic properties such as good covering power and pleasant application characteristics. The crystalline fullerene obtained by a standard synthetic procedure, as referenced in U.S. Pat. No. 5,612,021, does not possess the important properties of dissolved fullerene molecules. It has impeded access to living tissues, active centers of enzymes and other molecular biological objects, and it cannot pass through the cellular membranes to take part in biochemical processes of interest for cosmetics. Thus, U.S. Pat. No. 5,612,0211 essentially utilizes only the high optical density of the fullerene pigment, and fails to disclose the use of a fullerene as a biologically active component in cosmetics.

However, derivatives of fullerenes were shown to have biomedical applications. Chiang, et al. in U.S. Pat. Nos. 5,994,410 and 5,648,523 disclose chemical derivatives of fullerenes useful as free radical scavengers and the use thereof for treating free radical related medical conditions. Although the addition of functional groups to the fullerene molecules imparts the necessary solubility to the fullerene derivatives, the preparation of these derivatives inevitably results in a reduced number of double bonds and concomitant decrease in the ability of the fullerene molecules to scavenge free radicals. Moreover, functional groups create steric hindrances for interaction of fullerene cage double bonds with free radicals in solution, thus further decreasing specific reactivity of the fullerene.

Heretofore, no one has been successful in making a fullerene exhibiting pharmacological or biological activity without the addition of functional groups to the fullerene cage. However, the present inventors have found a means of dissolving fullerenes in non-toxic bio-compatible solvents without performing preliminary chemical derivatization of the fullerene moiety. They have found a way to maximize the useful chemical and biochemical reactivity of fullerenes. More specifically, they have found a method for solubilizing fullerenes by converting them into small molecular clusters, and subsequently using the solubilized clusters in cosmetic compositions. This takes advantage of the enhanced chemical reactivity of fullerene molecules and their small aggregates and the concomitant higher positive biological activity of interest for skin care and other cosmetic applications.

### SUMMARY OF THE INVENTION

Accordingly, the present invention is directed towards a cosmetic composition comprising a cosmetically effective amount of fullerene clusters in association with a cosmetic carrier.

Another aspect of the present invention is directed towards a method of preventing or reducing damage to healthy skin or mucous membrane of a mammal especially humans, by free radicals which comprises applying to the healthy skin or mucous membrane a cosmetic composition comprising a cosmetically effective amount of fullerene clusters.

Another aspect of the present invention is directed to a method of deactivating toxins in the skin or mucous membrane of mammals, especially humans, which method comprises applying an effective amount of fullerene clusters, in accordance with the present invention, to the skin of said mammal.

Yet another aspect of the present invention is directed to a method of treating antimicrobial or antiviral infection of skin and mucous membranes of a mammal by administering to the skin of said mammal an effective amount of fullerene clusters in the presence of solar light.

A still further aspect of the present invention is related to a method of human skin and mucous membrane protection against intense visible and UV irradiation, e.g. to prevent solar burns, by administering to the skin an effective amount of fullerene cluster.

### BRIEF DESCRIPTION OF THE DRAWINGS

FIG. 1 presents a space model of a hydrated fullerene molecule C₆₀(H₂O)₈₀ designed by Professor Martin Chaplin. The intact fullerene molecule (gray fused spheres for carbon atoms) is encased in the cavity formed by 80 water molecules, each water molecule being represented by a black sphere for an oxygen atom and two light gray spheres for hydrogen atoms. The cavity was formed by the withdrawal of a dodecahedron water cluster (H₂O)₂₀ from the water icosahedron (H₂O)₁₀₀. It is seen that directly over each fullerene hexagon a hydrogen atom from a water molecule is positioned, thus forming 20 hydrogen bonds between oxygen atoms and the pi-systems of the corresponding benzenoid rings. The accidental geometry correspondence results in unperturbed positioning of the inner 20 water molecules as well as the outer 60 molecules relative to original icosahedron structure, and therefore, an enhanced energy stabilization for C₆₀(H₂O)₈₀.

FIG. 2 is a schematic of the laboratory scale electrohydraulic impact chamber employed for fractionation of fullerite powder into the nanosize fullerene clusters. The chamber is filled to capacity with water and has the shape of an ellipsoid for rotation. Electric discharge through water is effected between two tungsten electrodes in a focal point of this ellipsoid, and the fullerite sample is positioned in another focus to be treated with the cumulative shock wave. The fullerite is suspended in a liquid inside a thin-walled plastic cell and thus separated from the outer-cell shock wave transmitting water medium. Alternatively, the fullerite suspension is continuously circulated in a closed contour, which includes a thin-walled plastic tube passing through the focal point. The liquid for suspending the fullerite is chosen from water, alcohols, natural oils, etc.

### DETAILED DESCRIPTION OF THE INVENTION

As used herein, the term “mammals” refers to any member of the class mammalia, including but not limited to, humans, dogs, cats, horses, pigs, cow, and the like. The preferred mammal is human.

Herein the term “cluster” has the general meaning defined in the chemical literature. A molecular cluster of fullerenes is a small assembly of fullerene molecules held together by Van der Waals forces. The size of this assembly may vary from a few fullerene molecules to about a billion. The internal structure of clusters is presumably close to that found in molecular crystals of fullerenes. Various shapes of clusters are conceivable, depending on cluster size and interaction with its environment. Clusters can be directly solvated by the solvent molecules, thus forming non-ideal solutions of fullerenes, close in nature to stable sols and suspensions. Clusters can form donor-acceptor complexes with certain organic molecules (ligands), which are soluble in polar solvents of interest and thus may impart fullerenes with solubility in these solvents as well. Both directly solvated and complexed fullerene clusters are utilized in the present invention.

The fullerene clusters described herein are prepared from fullerites (solid fullerenes). Examples of fullerites include solid forms of pure or mixed molecules C₆₀, C₇₀, C₈₄, C₉₆, and the like, that may incorporate crystallization solvents.

Fullerites or solutions or suspensions containing same are used as starting material for making the fullerene clusters of the present invention. Preferred fullerenes for this purpose are C₆₀ and C₇₀. The fullerenes are prepared in accordance with procedures known to one of ordinary skill in the art. For example, the fullerenes can be prepared by the methods described in Kratschmer, et al., Nature 347, 354 (1990), U.S. Pat. No. 5,273,729 to Howard, et al., U.S. Pat. No. 5,876,684 to Withers, et al., U.S. Pat. No. 5,227,038 to Smalley, et al., U.S. Pat. No. 5,300,203 to Smalley; and U.S. Pat. No. 5,556,517 to Smalley, U.S. Pat. No. 6,083,469 to Leftin, and U.S. Pat. No. 6,077,401 to Fields, et al., the contents of all of which are incorporated by reference.

As used herein, the term “fullerene” refers to a molecule containing an even number of carbon atoms arranged in a closed hollow cage. Fullerenes may contain even numbers of carbon atoms totaling from 20 to 500 or more. Fullerenes are not necessarily spherical. They may take the form of long tubular structures with hemispherical caps at each end of the tube. Hyperfullerene structures also exist wherein one structure is contained within a second larger structure. For generally spherical molecular structures, their hyperfullerenes resemble an onion layered structure. Tubular structures within larger structures are also possible. Fullerenes are more fully described in the literature. Examples of fullerenes include C₆₀, C₇₀, C₇₆, C₈₂, C₈₄, C₉₆, C₂₄₀, C₅₄₀, C₇₂₀ and the like. It is preferred that the fullerene has at least 60 carbon atoms. The preferred fullerenes are C₆₀ and C₇₀.

The fullerene clusters contemplated by the present invention may contain 1 type of fullerene or more than one type of fullerene. It is preferred that it contains 1 type of fullerene. For example, it may contain C₆₀ or C₇₀ or a mixture thereof, although it is preferred that it only contains one type of fullerene, either C₆₀ or C₇₀.

As described herein, the present invention is directed to making and using fullerenes in the form of molecular clusters. The formation of molecular clusters is exemplified by two methods.

In one method, the fullerites are mechanically disintegrated into separate small agglomerates of fullerene molecules (clusters). This is based on the mechanical fractionation of fullerite effected by hydraulic impact in various liquids, including water, alcohols, natural and synthetic oils, and the like.

The other method consists of solubilizing the fullerene solids in a liquid by complexing the fullerene clusters with water soluble polymers and/or porphyrins. In this case, the soluble complexes of fullerenes with polymers and/or porphyrins are shown to predominantly contain fullerenes in the form of small molecular clusters associated with polymer and porphyrin molecules as ligands. Examples of water soluble polymers useful in the formation of fullerene clusters using this method include polyvinylpyrrolidone (PVP), polyethylene glycol (PEG), polypropyleneglycol (PPG), copolymers of vinyl pyrrolidine, ethylene glycol, propylene glycol, and the like.

The water soluble polymers and the porphyrins are not chemically bound to the fullerenes by strong covalent bonds; instead they interact via Van der Waals forces The donor-acceptor bonding between the ligands and fullerene cluster in the complex does not significantly disturb the fullerene molecule electronic structure, i.e. all of the carbon-carbon double bonds of the fullerene cage are intact. Moreover, the donor-acceptor bonding has a reversible character ensuring both the delivery of a molecular cluster to the cell membrane through a water phase and the release of a single hydrated fullerene molecule that is capable of permeating the membrane. Since the carbon-carbon double bonds remain essentially intact and there are no steric hindrances invoked by functional group, the fullerene molecule thus delivered into tissues retains high chemical reactivity, which provides for the high biochemical activity of water soluble fullerene clusters, in particular, to act as antioxidants.

An exemplary procedure for preparing the water soluble fullerene complex with polymer and/or porphyrin ligands is outlined below. The fullerenes are dissolved in a non-polar organic solvent, such as benzene, toluene, or the like. The fullerene solution may contain one type or a mixture of more than one type of fullerene. A separate solution of the water soluble polymer(s) and/or porphyrin(s) is prepared in an appropriate organic solvent, such as chloroform, methylene chloride, ether or the like. The fullerene and ligand solutions are mixed in the argon gas filled reactor for a time sufficient for a reaction to proceed. For example, this may take from 30 minutes to one day, depending on the reagent concentration and the reaction temperature, which can range from about room temperature to the reflux temperature of the solvent mixture. The weight ratio of fullerene and polymer ranges from about 1:10 to about 1:10,000, more preferably from about 1:25 to about 1:250, and most preferably from about 1:50 to about 1:150; while the fullerene/porphyrin molar ratio ranges from about 1 to about 10; depending on the reagents and solvents chosen for the synthesis, and on the desired composition of the resulting complex. A colored product, e.g., a brown product, as in the case of PVP, is formed, indicative of the formation of the water soluble fullerene/ligand complex. After the reaction is over, the solvent is removed under vacuum, at about room temperature or slightly higher, for example, up to about 50° C.

After the solvent is removed, the cosmetic vehicle, e.g. water, alcohol, or more preferably a phosphate buffer with a pH of about 7.4 is added to the flask in sufficient amounts to dissolve the fullerene cluster and to form a solution. The dissolution of the fullerene complex in the vehicle may be accelerated through the use of heating, stirring, ultrasonic treatment or any other means known to one of ordinary skill in the art. The preferred method is 15-40 kHz and more preferably 20-40 kHz ultrasonic treatment at an intensity of about 15-50 W/cm² and more preferably 20-50 W/cm² for about 30 to about 600 seconds, and more preferably for about 30 to 300 seconds.

Optionally and preferably, the aqueous fullerene solution is filtered to remove impurities. Also, the fullerene solution may be concentrated to a predetermined level by reducing the solvent volume by evaporation.

Another method of forming the fullerene clusters is by mechanical dispersion of the fullerite (solid fullerene or a solid mixture of fullerenes) by subjecting the fullerite to mechanochemical treatment. This treatment is effected in a liquid medium to split the fullerene to nanosize clusters. Preferably, the fullerites are reduced to the size of about 5 to about 50 nm and more preferably from about 5 to about 15 nm. Any method known to one of ordinary skill in the art can be used to convert the fullerite to nanosize clusters. Examples include electrohydraulic impact, ultrasonic- and acoustic wave treatments, in the power range from about 0.01 to about 100 kW. The preferred method is electrohydraulic (EH) impact, as it generally produces smaller clusters having greater efficiency.

The essence of the EH effect consists of the production of high magnitude and gradient local dynamic hydraulic pressure during the pulse of a high voltage electric discharge inside a liquid. This kind of dynamic pressure is efficient at cracking solids, and thus forms fine dispersions of solids in the liquid. Examples of an apparatus that may be useful for disassociating fullerenes by EH impact are described, for example, in U.S. Pat. Nos. 6,254,764 to Babington, et al., 5,868,919 to Babington, et al. and 4,917,785 to Juvan, et al., the contents of all of which are incorporated by reference.

There are several advantages of the EH effect technology for comminution of fullerites.

When electrical energy is suddenly released into a liquid in the form of a spark discharge between two electrodes, an intense mechanical shock wave propagating at supersonic velocity is created. The shock wave consists of two zones. The steep leading edge of the shock wave represents the compression zone, which is followed by the rarefaction zone, where the pressure falls off from its peak value to a small negative value and then returns to its pre-shock value. The rarefaction zone is travelling faster than the compression zone and the entire shock wave gradually decreases in amplitude as it travels. Both zones perform the work on distortion of a solid particle met by the shock wave in the liquid. The distortion can eventually lead to particle fracture, depending on particle mechanical properties and shock wave parameters. When a shock wave enters a solid body it causes dilatational or longitudinal stress and shear or transverse stress. The stress a is linearly related to the density of the solid p, the wave velocity C and the solid particle element velocity V through the equation: σ=ρCV. This relationship applies to both longitudinal and shear waves. The shock wave velocity at EH impact caused by electric discharge could reach about 18 km/s, which exceeds the velocity of sound in water (˜1.5 km/s) by an order of magnitude. It also is much larger than the velocity of shock waves (˜2 km/s) generated by implosion of cavitation microbubbles during ultrasonic treatment of water. Accordingly, the magnitude of stresses developed during EH impact greatly exceeds that from ultrasonic treatment. Therefore, the EH impact is much more effective in comminution of fullerite particles suspended in water, compared to other treatments capable of generating shock waves of lower velocity.

As the tensile strength of fullerite is much less than the compression strength, the disintegration of fullerite particles is likely to occur mainly in the rarefaction zone of a shock wave, terminating with the so called tensile or negative phase of a shock wave. A negative peak pressure up to 10 MPa and of a total duration of a few microseconds is developed in the tensile phase of a shock wave in water. This tensile load is transmitted over the fullerite particle, causing it to crack along the plane parallel to the dilatational wave front. The distortion of a fullerite particle due to the shear strain splits the particle in normal to dilatational wave direction. This process significantly contributes to particle fragmentation, as the shear strength of fullerite is low while the transverse wave velocity is comparable to that of the longitudinal wave.

These main fragmentation processes are complemented by fullerite particle fragmentation caused by secondary shock waves and liquid microjets produced by the collapse of cavitation bubbles. This fullerite particle fragmentation caused by secondary shock waves and liquid microjets generally cannot form the smaller particle sizes found in the fullerene clusters. When the peak amplitude of the negative pressure in the tensile phase of a shock wave exceeds about 1 MPa, transient cavitation bubbles of about 100 micrometer in diameter are induced in water. The collapse or implosion of cavitation microbubbles takes less than a microsecond and causes the gas pressure and temperature inside the cavity to jump to about 150 MPa and 5,500 C, correspondingly. Hot spots that are thus created are capable of producing secondary shock waves, which are still powerful enough to demolish solid particles suspended in water. The implosion of a bubble formed near a solid surface is asymmetric, so that a jet of liqiuid about 10 micrometers in diameter and 50 micrometers in length is expelled from the collapsing cavity. This jet moves toward the surface at roughly 100 meters per second. The microjets impinge onto the fullerite surface and cause fullerite particle fragmentation. This process produces fragments commensurate with the jet size, that is, in the micrometer range, but it is most likely incapable of producing nanometer size particles. This is further supported by the fact that cavitation bubbles of the same nature and function are produced in ultrasonic treatment of fullerite suspensions, and the characteristic size of particles produced is appreciably larger than that obtained during EH impact treatment, with similar energy input into the liquid in both methods.

Thus, the EH technique has important advantages over more common ultrasonic and acoustic wave treatments, rendering it preferable for producing nanosize clusters of fullerenes. It can be easily scaled up. Facile control of key features of EH process is ensured in very wide range of parameters. Moreover, the magnitude of many parameters important for the fractionation process are achieved by this EH technique but inaccessible in other methods, as can be inferred from the following listing of EH shock wave properties and EH devices features:
Typical shock wave front rise time—10-100 ns
Total pulse duration—100 ns-10 μs
Maximum pressure in the hot plasma channel—1-3 GPa
Typical positive peak pressure on the sample—100 MPa
Typical negative peak pressure on the sample —1-10 MPa
Maximum temperature in the hot plasma channel—15,000-40,000 K
Maximum initial shock wave velocity—10-18 km/s
Pulse energy—0.5 CU²=˜1-100 kJ
Specific energy per pulse—˜5 J/cm³ of liquid in typical fractionation reactor
Specific power in the pulse—2 MW/cm³ of liquid
Efficiency of conversion of electrical energy into mechanical—˜50%
Discharge capacitor voltage—3-100 kV
Pulse discharge current—10-1000 kA
Electrical capacity—3-1000 pF
Inductance of discharge circuit—5-50 nH
Practical rate of pulse repetition—0.2-20 pulse/s
No impurities are introduced into the product in the EH apparatus.
For the sake of comparison, some features of ultrasonic treatment technique are presented below:
Typical compression wave rise time—50 μs
Maximum positive peak pressure on the sample—5 MPa (Typical 1-2 MPa)
Maximum negative peak pressure on the sample—5 MPa (Typical 1-2 MPa)
Frequency of compression-dilatation cycles—20-40 kHz
Ultrasound wave velocity in water—1.5 km/s
Cavitation bubble diameter—60-200 μm
Cavitation bubble growth time—100-400 μs
Cavitation bubble collapse time—100 ns-1 μs
Gas pressure in the collapsed cavitation bubble—150 MPa
Gas temperature in the collapsed cavitation bubble—5,000 K
Liquid temperature near collapsed bubble—2,100 K
Efficiency of conversion of electrical energy into mechanical—˜50%
Yield of OH radicals G˜1×10⁻¹⁰ mol/J; Number of OH radicals produced˜1×10⁶/cycle
Continuous power input—50 W-5 kW
Surface density of power input—1-100 W/cm²
Specific power for continuous treatment—1-100 W/cm³ of liquid

Some physical properties of solid fullerite C₆₀ are presented below to enable assessment of its behavior under conditions of the EH and ultrasonic treatments:
Mass density—1.72 g/cm³
Bulk modulus—14 GPa
Longitudinal velocity of sound—3.6 km/s
Transversal velocity of sound—2.1 km/s
Thermal conductivity (300 K—0.4 W/mK
Phonon mean free path—5.0 nm
Compressibility (−dV/dP)—6.9×10⁻¹² cm²/dyn
Volume coefficient of thermal expansion—6.2×10⁻⁵ cm³/K

It is clear from the data above that the EH technique is more efficient in fractionating the solid fullerite than ultrasonic treatment, and in particular more energy efficient and productive. However, ultrasonic treatment of great duration can also provide fullerene clusters in the form of stable colloidal solutions, which could be barely distinguished visually from the product of EH technique. Therefore, ultrasonic treatment is another method for fullerene cluster production, as it can be efficiently employed in auxiliary operations such as re-dispersion of the dried fullerene cluster product in liquids, mixing of fullerene clusters with cosmetic vehicles, and the like. However, as previously stated, the more preferred method of forming fullerene clusters is best accomplished by the EH technique.

The data on the structure of fullerene clusters is scarce. Without being bound to a particular hypothesis or instrumental observation, the probable structure of clusters and composition of solutions obtained can be presented as follows. Microcrystalline fullerite is mechanically loose and becomes disintegrated into nanosize clusters when adequate tensile or shear strain is applied. Appropriately prepared fullerene clusters do not precipitate from water or any other liquid solution of interest for a period generally exceeding several months. The reason for this stability is not yet definitively established, and may be different in different solvents.

The water soluble polymer(s) and/or porphyrin(s) stabilize the fullerene clusters. Without wishing to be bound, it is believed that the stability can be explained as follows. Fullerene clusters, having sizes as small as individual molecules, have attractive interactions with electron-donating groups of polymers and/or porphyrins, and thus form a complex which is soluble in water and other solvents due to the presence of polar groups of the polymer and/or porphyrin that presumably do not bond to the fullerene. Formation of such complexes has been confirmed by spectral techniques. Occurrence of fullerenes in cluster form in these complexes is supported from the appearance of absorption bands in their electronic spectra in the visible range that are characteristic of fullerene molecules in close Van der Waals contact to each other. This characteristic absorption generally is more pronounced in the short-wave region of the visible range. Moreover, it imparts to solutions of fullerene cluster complexes a color that is different than would be present if the fullerene were dissolved in solution. For example, a solution of fullerene 60 cluster complexes have a yellow to brown coloration, instead of the violet color of true solutions of fullerene C₆₀ containing separate molecules.

Similar spectral features and color changes are intrinsic to fullerene, e.g., C₆₀, solutions in unsaturated fatty acids and natural and synthetic oils that contain double C═C bonds in their structure. This indicates the occurrence of fullerene C₆₀ in the cluster form in these solvents as well.

Without wishing to be bound, it is believed that the strong polarizablility of fullerenes, such as C₆₀, coupled with the polarizable double C═C bonds in ligands, is considered to be responsible for the enhanced attraction between ligands and fullerene clusters, which results in the formation of a soluble complex. This complex prevents aggregation of clusters and thus precipitation of solid fullerenes, e.g., C₆₀, from the solution. The solubility of fullerenes in natural oils reaches dozens of milligrams per milliliter, and thus exceeds the solubility in common aromatic solvents that are among the best solvents for fullerenes. The interaction of fullerene molecules with molecules of natural oils and aromatic solvents is realized mainly through the polarization forces described hereinabove. It is reasonable to assume that the total strength of this interaction, and hence the solubility value, would be roughly proportional to the number of double C═C bonds present in the solvent and available in contact with fullerene molecule. As this number is much higher for an aromatic solvent than for a natural oil, the solubility in an aromatic solvent should be much higher as well. Since the opposite occurs, this can be considered an indication that fullerene solutions in natural oils are actually stable sols of fullerene clusters rather than true solutions.

The stability of fullerene clusters in pure water is different. Without wishing to be bound, it is believed that negatively charged fullerene clusters dominate in water solution. The most likely reason for the appearance of negative charge seems to be dissociation of hydroxyl groups covalently bound to the fullerene cage. The acidity of these hydroxyl groups is likely much higher than in water molecules because of the extremely high electron affinity of the fullerene molecule(e.g., 2.65 eV for C₆₀, the smallest fullerene). This ensures facile release of protons into water in pH ranges down to about 3, and the concomitant formation of negatively charged particles. The attached hydroxyl groups originate from the attack of hydroxyl radicals on the double bonds of a fullerene.

The main source of hydroxyl radicals in water solutions is the collapse of cavitation bubbles produced during ultrasonic treatment or shock wave passage. The rate constant for the reaction of hydroxyl radicals with organic molecules, including compounds with double C═C bonds, is close to the diffusion-controlled value; therefore, for example, ultrasonic treatment is used in water purification systems to get rid of trace amounts of phenols and other organic contaminants. The reaction should be very efficient in the case of fullerene clusters as well, thus providing the fullerene molecules at the surface of clusters with some hydroxyl groups.

These few ionogenic hydroxyl groups are necessary to create a double electric layer at the surface of a fullerene cluster and thus stabilize the colloid particles against coalescence. The quantity of hydroxyl groups present can vary depending on the cluster size and the pH of the medium, and may be too small to be detected. Nevertheless, there exists an instrumental technique capable of revealing the presence of such small quantities of hydroxyl groups. Specifically, a broad light absorption is developed near the 450 nm wavelength in the spectra of fullerene e.g., C₆₀, solubilized in water by ultrasonic or by EH impact treatment. It is generally attributed to the disturbance of fullerene molecule electronic structure, caused by attachment of functional groups, in particular, hydroxyl groups. This absorption at 450 nm is commonly much more intense than what could be expected from that originating from inter-fullerene Van der Waals interactions described above for the case of fullerene cluster complexes with various non-covalently bound ligands. The enhanced absorption at 450 mm thus provides additional evidence that fullerene clusters with hydroxylated fullerene molecules (fullerols) at their surface are formed.

In this structural model, without wishing to be bound, it is presumed that the inner fullerene molecules in the cluster are largely intact and held together by inter-fullerene Van der Waals forces. However, the incorporation of some low-order fullerols into the cluster inner space is also possible, as fullerols can also form crystalline Van der Waals structures. Without wishing to be bound, it is believed that the fullerene clusters so structured could be transported through the intercellular medium to the cell membrane.

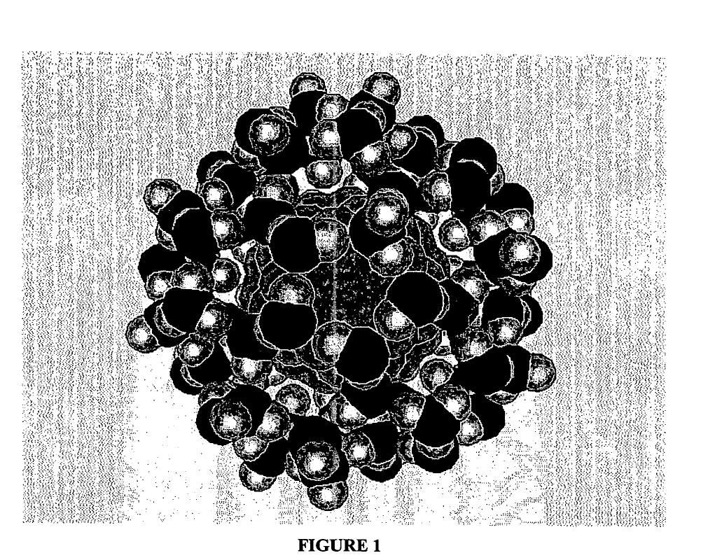

There are experimental observations that confirm that fullerene clusters exist in a dynamic equilibrium with hydrated single fullerene moieties. The thus produced hydrated fullerene molecules could be either fullerols or intact fullerene cages. The existence of the latter has been postulated in the literature, and a molecular model for the solvation of an intact fullerene cage such as C₆₀ by water molecules has been designed (FIG. 1). Water in the liquid state possesses a virtual crystalline structure. In the molecular model, the C₆₀ fullerene is located inside a hollow icosahedron composed of 80 water molecules. The inner twenty water molecules are ideally situated to form OH. . . p hydrogen bonds with each of the twenty 6-membered rings in the fullerene C₆₀, by positioning directly over these rings, which is the optimum position for a hydrogen bond with a benzene molecule. These 20 water molecules are linked through the 60 fully hydrogen bonded water molecules from the outer shell of the icosahedron. It is also believed that the stability of hydrated low-order fullerols would also benefit from this fairly unique structural coincidence of the cavity and fullerene molecule.

Without wishing to be bound, it is believed that the hydrated single-cage low-order fullerols and hydrated pristine fullerene molecules penetrate the cell membranes and take part in the cell metabolism, while larger fullerene clusters are deemed merely responsible for fullerene biochemical activity outside of the cell. The equilibrium co-existence of these soluble forms of fullerenes can be used for the rationalization of a variety of known useful functions of fullerenes in cosmetic applications, and constitute a basis for designing new cosmetic systems.

The clusters of fullerenes formed herein may be placed in aqueous solutions or organic solvents such as alcohols and oils. For the EH dispersion and ultrasonic dispersion of fullerene-containing substances with the purpose of subsequent use in cosmetic products, the most appropriate solvents are water or alcohol such as alcohol containing 1-6 carbon atoms as well as natural and synthetic oils. A natural oil, as defined herein, is a complex mixture of various natural compounds with one or several double bonds, and C═O and C—OH groups. Preferably these are polyunsaturated fatty acids—from about 16 to about 38 carbon atoms. The flexibility of hydrocarbon fragments located between functional groups in the polyunsaturated fatty acids enables simultaneous donor-acceptor type interaction of several of their functional groups of the polyunsaturated fatty acids with a fullerene cluster, which interaction is responsible for the high solubility of fullerences in oils. To prevent excessive formation of free radicals during EH and ultrasonic dispersion treatments of fullerites in oils, the applied power density should be reduced to approximately one half of the optimal value for water and alcohol liquid media. For example, the ultrasonic dispersion treatment at 10-18 W/cm² power density level is optimal for dissolution of fullerites in various oils, and does not cause either deterioration of oil quality or chemical bonding of the polyunsaturated fatty acid molecules to fullerenes. The fullerene cluster solutions thus obtained in oils can be used for preparation of cosmetic compositions without further purification.

The fullerene clusters in the composition of the present invention may be associated with water soluble polymers and/or porphyrins. Examples of water soluble polymers that can associate with the fullerene clusters include polyvinyl pyrrolidone, polyethylene glycol, polypropylene glycol, copolymers of vinyl pyrrolidone, ethylene glycol, propylene glycol and the like. Alternatively, or in combination with the polymers and/or water soluble polymers, the fullerene cluster of the present invention may be associated with a terpene.

Terpenes, are chemical compounds of the simplest formula C₁₀H₁₆. However it is problemed that the terpene is an unsaturated unsaturated acryclic and cyclic hydrocarbon of the formula (C₅H₈)ₙ where n>2. Terpene are components of ether oils. Terepenes are natural hydrocarbons and are contained in plants. Terpenes are composed of isoprene units. Isoprene (2-methylbutadiene-1,3), CH₂═C(CH₃)—CH═CH₂, is a component of such natural substances as caroteniodes (e.g., β-carotene) and phythol, a part of chlorophyll. The ether oils, for example, camphor oil, contain not only terpenes, but also terpenes derivatives (alcohols, aldehydes, ketones, esters and ethers).

Terpenes are capable of dissolving appreciable amounts of fullerenes. For example, the solubility of C₆₀ in terpenes or the aforementioned derivatives varies in the range from about 3 to about 20 mg/ml. The dissolution of fullerenes in terpenes is facilitated by implementing the same physical activation means, which are described herein for “dissolution of fullerenes in water, oils, and unsaturated acids. These means include, among others, ultrasonic treatment and electrohydraulic impact technique being applied to the mixture of fullerene and liquid terpenes, as well as common supplementary treatments, including elevated temperature, stirring, maceration, ketones attrition milling, and the like.

Terpene derivatives such as alcohols, aldehydes, and lower alkyl (C₁-C₆) esters leave alkyl (C₁-C₆) ethers or acids of terpene may also be associated with the fullerene clusters. The terpenes as well as the terpene moiety in the derivative contain at least 10 carbon atoms wherein m is at least 1, and is preferably no more than 100 and more preferably not more than 10, and most preferably is 1. Examples include
Geraniol, C₁₀H₁₇OH (alcohol with the scent of roses),
Citrinellol, C₁₀H₁₉OH (alcohol with a tender scent of roses),
Citrel, C₉H₁₅CCHO (aldehyde with lemon fragrance),
Linalyl acetate, CH₃COOC₁₀H₁₇ (ester with lavenda scent),
Menthol, C₁₀H₁₉OH, (with mint scent)
Terpineol, C_{10,}H_{17,}OH (with lilac scent),

These terpenes solvents are widely used in perfumery and cosmetics as fragrances. Moreover, some of them, for example, menthol, has strong bactericide properties. This reduces ans/or exclude other conservants in appropriate fullerene creams. Conservants are often allergic agents.

The highly solubility of fullerenes in terpenes and their derivatives allows efficient use of these solvents in fullerene cosmetics (e.g. allows one to achieve sufficiently high concentration of fullerenes in creams). Fullerenes are present in these solutions in cluster form. That is, fullerenes are solubilized as clusters in these terpene solvents and eventually in the cream or ointment or rouge, or whatever other fullerene cosmetics is utilized. The fullerenes therein are clusters suspended in the terpene solvent in a form close in essence to a (nano) colloidal solution.

Solubilized terpenes (fullerene clusters) can be easily incorporated into a cream or other cosmetic means by general operations well known in the art to obtain cosmetics with excellent quality.

The commentary herein regarding fullerene clusters in association with oils and unsaturated acids is equally applicable to terpene.

The fullerene clusters in the present composition are combined with cosmetically useful materials to form cosmetic compositions. Cosmetically useful materials include, but are not limited to, antioxidants, binders, bulking agents, chelating agents, colorants, emollients, emulsion stabilizers, film formers, fillers, fragrance components, gelling agents, hair conditioning agents, hair fixitives, humectants, plasticizers, preservatives, skin conditioning agents, solvents, sunscreen agents, surfactants, ultraviolet light absorbers, viscosity controlling agents and waxes. Various other cosmetically useful materials can be found in the CTFS Cosmetic Ingredient Handbook, J. M. Nikitakis, Ed., 1^{st} Edition, pages 51-101 (1988), the disclosure of which is hereby incorporated by reference.

Cosmetic compositions of the invention include compositions that may contain a wax.

The fullerene clusters prepared in accordance with the present invention are associated with a cosmetically acceptable carrier. The vehicle may be an aqueous solution, e.g. water; or a polar organic solvent, alcohol, e.g. ethanol or another polar-solvent; a natural or synthetic oil; an oil-in-water emulsion; or a water-in-oil emulsion; or a wax; and the like. The carrier should obviously be non-toxic. The preferred carrier of the compositions of the present invention is an aqueous solvent.

Depending upon its function, the cosmetic compositions of the present invention may be provided in various forms, such as solutions (lotion type compositions), thickened solutions, gels, ointments, emulsions (cream, milks), vesicular dispersions, powders, dense powders, pastes or solid agents. More specifically, the cosmetic compositions of the present invention may be dispersed in various forms, including, but not limited to, blushers, creams (including face creams, hand creams, moisturizing creams and sunscreen creams), cream powders, eye liners, eye shadows, eyebrow pencils, foundations, lotions, mascaras, microemulsions, ointments, pomades and rouges. They may also be packaged in pressure packs containing a propelling agent permitting application in the form of foams or sprays.

The fullerene clusters described hereinabove may be added to various type of cosmetic compositions. For example, they may be added to pharmaceutical compositions which protect the human epidermis, hair and mucous membranes; makeup compositions for the skin and superficial body growths; compositions for buccodental use such as dentifrices or ophthalmic compositions such as collegria, and the like.

The fullerene clusters of the present invention are present in cosmetically effective amounts or if used in pharmaceutical compositions, in therapeutically effective amounts, as described herein. Preferably, they are present in the compositions of the present invention from about 0.01 to about 50% by weight of the total composition, and more preferably from about 0.02 to about 10% and most preferably from about 0.05 to about 2% by weight.

When the cosmetic compositions according to the present invention are used for protecting the hair, they may be provided in the form of shampoos, lotions, or gels; or compositions to be rinsed, or to be applied before or after shampooing, before or after dyeing or bleaching or before, during, or after permanent waving of the hair or of hair straightening treatments. It may also be provided in the form of hair styling or treating lotions or gels, lotions or gels for blow drying or hair setting, hair lacquers, compositions for permanent waving or hair straightening, or compositions for dyeing or bleaching the hair.

When the composition of the present invention is used as make-up products for the eyelashes, the eyebrows, or the skin, it is provided in the form of foundations, lipsticks, eyeshadows, blushers, eyeliners, mascaras, or creams for treating the epidermis.

When the composition of the present invention is used as a pharmaceutical composition, e.g. a sanitizing composition, a composition for reducing the appearance of lines and wrinkles, a moisturizer or the like, it may be provided preferably in the form of an emulsion, such as milk or cream, gel, lotion, ointment, or vesicular dispersion, and may contain pharmaceutically active ingredients.

The cosmetic compositions may contain natural or synthetic waxes. Natural waxes may be of animal origins, such as lanolin, beeswax, spermaceti or lanolin derivatives, such as lanolin alcohols, hydrogenated or acetylated lanolin, fatty acids of lanolin or acetylated lanolin alcohol, or of vegetable origin, such as carnauba, candelilla, kapok, rice, hydrogenated jojoba, alfa or yapan wax or cork fiber, sugar cane waxes, cocoa butter or the like. Alternatively, mineral waxes may be present in the cosmetic composition, such as paraffin, montan, lignite, petrolatum, petrolatum waxes or microcrystalline waxes, ceresin, or ozokerite. Examples of synthetic waxes that may be used include polyethylene waxes, the waxes obtained by Fischer-Tropsch synthesis and the linear esters resulting from the reaction of a saturated C₁₀-C₄₀ carboxylic acid and of a saturated C₁₀-C₄₀ alcohol, such as myristyl myristate. Other waxes that may be used include calcium lanolates or stearates, or hydrogenated coconut oil, and the like.

The cosmetic compositions may contain unmodified or modified oils of vegetable or animal origin, for example, sweet almond oil, avocado oil, castor oil, olive oil, jojoba oil, sunflower oil, wheat germ oil, sesame oil, groundnut oil, grapeseed oil, soybean oil, safflower oil, coconut oil, maize oil, hazelnut oil, karite butter, palm oil, apricot kernel oil, calophyllum oil or perhydrosqualine. Moreover, the oil phase may be a mineral oil, for example, liquid paraffin, liquid petrolatum and the like. The oil may be a synthetic oil, e.g. fatty acid esters, such as isopropyl myristate, isopropyl palmitate, 2-ethylhexyl palmitate, penicillin oil (stearyl octonate), unsaturated fatty acids, such as oleic, palmetic, stearic, behenic, linoleic, lanoleic acid or volatile or non-volatile isoparaffins, e.g. C₈-C₁₆ isoparaffins, and the like.

Moreover, the oil may be C₁₂-C₁₈ fatty alcohols, such as oleyl alcohol, cetyl alcohol and stearyl alcohol and the like.

If present as an emulsion, the emulsified composition of the present invention includes an oil phase and an aqueous phase. The oil phase preferably is present in an amount ranging from about 1% to about 75% by weight, based on the total weight of the composition, more preferably from about 5% to about 60% by weight and most preferably from about 40% to about 60% by weight.

The aqueous phase may include adjuvants commonly used in aqueous gels and cosmetic emulsions. The aqueous phase may be present from about 0.5% to about 20% by weight and may also include a lower C₂-C₆ monoalcohol and/or a polyol, such as glycerol, butylene glycol, isoprene glycol, propylene glycol, ethylene glycol, and the like.

Emulsifiers can be used to assist in preparing an emulsified cosmetic composition. Any cosmetically acceptable emulsifier can be used in an amount that provides the desired emulsifying effect. The emulsifiers are generally selected from known soaps and surfactants. Preferably, the emulsifiers are selected from stearic acid, sorbitan sesquinoleate, polyethylene glycol (PEG-30), dipolyhydroxystearate, lecithin, magnesium stearate, and derivatives and mixtures thereof. The emulsifiers preferably are used in amounts ranging from about 0.5 to about 30% by weight, based on the total weight of the composition, more preferably from about 1% to about 12% by weight and more preferably from about 4% to about 8% by weight.

A thickening agent may also be present. Any thickening agent normally used in cosmetics can be used. Examples include modified clays, such as modified magnesium silicate (bentone gel VS38 from Rheox), hectoxite modified by distearyldimethylammonium chloride (benetone 38 CE from Rheox), cross-linked polyacrylic acids and guar gums and celluloses which may or may not be modified.

The composition of the present invention can also contain a film-forming compound. Film forming compounds can be chosen from polymers in aqueous dispersions, such as, for example, acrylic, polyester and/or polyurethane polymers in aqueous dispersion, e.g. partially neutralized vinyl acetate/vinyl p-tert-butyl/benzoate/crotonic acid copolymer in aqueous dispersion.

The compositions of the present invention may also contain coloring agents. These coloring agents can be either inorganic or organic pigments which are generally insoluble in aqueous and organic media or dyes which are, soluble in aqueous or organic matter.

A lubricity agent is another optional component. A lubricity agent generally aids in creating a soft and smooth feel of the composition to the hands. Examples include benzoic acid esters of C₁₂-C₁₅ alcohols, volatile silicones such as cyclomethicones, dimethicones and derivatives thereof. Examples of silane containing compounds useful in the present invention include cyclomethicone tetramer and pentamer (available as Dow Corning 244 or 245 Fluids) or non-volatile silicones such as stearyl dimethicone (available as Dow Corning 2503 Cosmetic wax), or derivatives thereof, such as cyclomethiconepolyol, dimethiconepolyol, cetyl dimethicone copolyol, phenyl methicone, phenyl trimethicone, and the like. Those skilled in the art will also appreciate that the silicone containing compounds may also function as an emulsifier.

The silicone containing compounds are preferably present in an amount ranging from about 1% to about 50% by weight, based on the total weight of the composition, more preferably from about 5% to about 30% by weight and most preferably from about 10% to about 25% by weight.

A moisturizing agent may also be added to the cosmetic composition. A “moisturizing agent” is an agent that provides a moisturizing effect to the skin, e.g. humectants. The moisturizing agent is present in a moisturizing effective amount. Examples of moisturizing agents include glycerin, butylene glycol, propylene glycol, sorbitol, sodium PCA, glucam E-10, glucam E-20, and the like. Preferably the moisturizing agent is present in an amount ranging from about 0.1% to 10% by weight of the total composition, and more preferably from about 0.1 to about 5% by weight and most preferably from about 1% to about 5% by weight.

Additionally, emollients may optionally be present. It is preferred that the composition contains one or more emollients. Emollients are used to impart a smooth and soft feeling to the skin surface. This is effected without measurably affecting the skin hydration level and/or the skin liquid barrier. Examples of emollients include vegetable triglycerides, such as avocado oil, olive oil, sunflower oil, organic acid esters such as sorbitan oleate, myristyl myristate, isopropyl myristate, glycol oleate, mineral oil, glycerin, petrolatum, petroleum jelly, and the like. Emollients may be present in an amount ranging from about 1% to about 50% of the composition, more preferably for about 5% to about 40% and most preferably from about 10% to about 25% of the composition.

Additionally, antioxidants may also be present in the cosmetic composition of the present invention. The antioxidant may be natural or synthetic. Examples include tocopherol, Vitamin E derivatives, e.g. Vitamin E linoleate, Vitamin E, Vitamin E POE succinate, Vitamin E acetate, ascorbic acid, ascorbyl palmitate and ascorbyl-PMG. It is used in effective amounts to neutralize harmful oxidants, e.g. singlet oxygen. Preferably, it is present within the range of about 0.1% to about 5% by weight and more preferably from about 0.5% to 1% by weight.

The cosmetic composition may also contain ingredients that reduce the oily feel of emollients in the composition, e.g., cream, known in the art, e.g., PPG-2-myristyl ether propinate, isopropyl palmitate and the like. If present, they are present in an amount from about 1% to about 30% and more preferably from about 5% to about 25% and most preferably about 10% to about 20% by weight of the composition.

A pH adjusting agent is commonly used to adjust the acidity of the composition to a desirable range, preferably to a pH of about 6 to 8. An example is aminomethylpropanol. This pH adjusting agent is added in an amount effective to change the pH of the composition to the desired pH range. Preferably, it is present in an amount that is less than 0.4% by weight of the total composition.

A sunscreen agent may also optionally be present. The expression “sunscreen agent” denotes sunscreen agents that are useful in absorbing, screening or preventing ultraviolet rays from penetrating the skin. Preferably, the sunscreen agent is titanium dioxide or zinc oxide and more preferably the sunscreen agent is coated with a polymeric material or other cosmetically acceptable coating. Examples of sunscreen agents are micronized titanium dioxide coated with aluminum stearate, or C₉-C₁₅ polyfluoroalkyl phosphate, polymer coated zinc oxide, aminobenzoic acid (PABA) and its esters, benzophenone-3, octyl salicylate, menthyl anthranilate, phenylbenzimidazole sulfonic acid and the like.

Specifically, the sunscreen agent provides a protection factor (SPF) in the range of 2-12 for minimal, 12-30 for moderate and above 30 for high sun protection.

The balance of the composition is comprised of customary additives selected from auxiliaries, fillers, organic solvents (such as alcohols and oils), buffers, perfumes, self-tanning agents (e.g. dihydroxy acetone) and the like and other cosmetically acceptable carriers and fillers. Other conventional additives, that can be used in the present cosmetic compositions, include dispersants and preservatives. The amount of conventional additives typically range from about 0 to about 20% by weight based on the total weight of the composition, preferably from about 1% to about 15% by weight and more preferably from about 4% to about 10% by weight.

It will be appreciated that the remaining percentage or balance of the composition is water. Water acts as a vehicle to ensure even distribution of the composition to the skin. It is preferred that the water utilized is deionized or distilled water and the amount of water is typically anywhere from about 10% to about 80% by weight, and most preferably from about 20% to about 40% by weight.

In a preferred embodiment, the cosmetic composition of the present invention is a cream or lotion or ointment, but most preferably a cream. The carrier is preferably water. The preferred cream composition is formulated as an oil in water emulsion that contains the necessary common cosmetic ingredients known to one skilled in the art for ensuring high user properties, in addition to the fullerene cluster component. In one preferred embodiment, these ingredients include petrolatum, especially white petrolatum, preferably in an amount ranging from about 0.5% to about 1% by weight of the composition, lanolin alcohol preferably in an amount ranging from about 0.5% to about 1% by weight of the composition, PPG-2-myristyl ether propionate, preferably in an amount ranging from about 3% to about 6% by weight of the composition, mineral oil, preferably in an amount ranging from about 5% to about 10% by weight of the composition, triethanolamine, preferably in an amount ranging from about 0.2% to about 0.8% by weight of the composition, glycerol, preferably distilled, and preferably in an amount ranging from about 2% to about 4% by weight of the composition, stearic acid, preferably in an amount ranging from about 15% to about 20% of the composition, isopropyl palmitate preferably in the amount of about 10% to about 15% by weight of the composition and the remaining amount is water. Fragrances, perfumes, vitamins and other common ingredients known to one of ordinary skill in the art normally present in cosmetic or pharmaceutical compositions such as those outlined hereinabove as well as antioxidants and coloring agents, depending on the specific cream formulation desired, may also be present.

The cosmetic compositions of the present invention containing fullerene clusters, prepared as described hereinabove have several utilities. First, they produce a marked rejuvenating effect on the skin, described in some preferred embodiments of this invention.

Inasmuch as double bonds in the fullerenes are readily available for reduction-oxidation (redox) reactions, the cosmetic compositions of the present invention have strong anti-oxidant properties that are likely, depending on the fullerene clusters capacity, to induce strong negative catalysis that blocks chain radical oxidation processes. It is believed that free radicals produced during metabolic reactions in the skin cells are one of the major factors of skin aging. The cosmetic compositions of the present invention, when applied to the skin in effective amounts, prevent free-radical damage to cells, thus rejuvenating the skin, and in particular reducing the appearance of wrinkles.

The compositions of the invention can be applied to the skin on a daily basis and more preferably twice daily. In order to achieve pronounced and stable rejuvenating effects, they should be applied regularly for weeks or months, although the effectiveness of the compositions usually gives visible results starting from the first few applications. The rate and duration of application can be varied, however, depending on the concentration of effective ingredients. Skilled artisans are capable of designing a suitable application regimen to provide the requisite rejuvenating effect.

Mitochondria in the human body produce kilograms of free radicals annually in the process of normal cellular metabolism. The capacity of cells to defend against oxidation damage initiated by free radicals reduces with time, which is the cause of cellular and organism malfunctions generally recognized as normal aging phenomena. Accelerated aging occurs when additional sources of free radicals appear. Some atmospheric pollutants, such as sulfur dioxide, ozone and nitrogen oxides, possess free radical-initiating activity, and thus represent additional exogenous sources of free radicals. Living cells that are in permanent contact with the external medium (especially the skin, the scalp and certain mucous membranes) are particularly subjected to toxic effects of gaseous pollutants. These effects result in accelerated aging of the skin, manifested by a complexion lacking brightness and the premature formation of wrinkles, and also in a decrease in the vitality and a dull appearance of the hair. The cosmetic compositions of the invention are effective in reducing accelerated aging of the skin, and in revitalizing hair.

Another exogenous source of free radicals is brought to the skin, hair and mucous membranes by exposure to ultraviolet rays. Besides being capable of causing aging phenomena, UV rays are currently considered a major factor in inducing skin tumors. Radical species formed by UV radiation are particularly considered culprits in the oxidation of skin lipids, and it is thought that lipid peroxides are one of the factors which trigger photocarcinogenesis. It is known in particular that the induction of ornithine decarboxylase (abbreviated ODC) is an early marker for skin tumors, and that organic peroxides are capable of inducing the formation of ODC in the epidermis; see R. L. Binder et al., Carcinogensia, Vol. 10, No. 12, 2351-2357 (1989). Some fullerene compounds are capable of reducing the level of ODC in the skin. Fullerene clusters possess the same property due to their ability to deliver active fullerene molecules into the skin cells. Thus the cosmetic compositions of the invention can make it possible, in addition, to prevent or limit the risks of skin cancers. This has been confirmed by the positive changes in the skin superficial body growth, observed in experiments on topical application of a composition of the present invention to the skin superficial body.

The fullerene containing cosmetics of the present invention destroy bacterial toxins on the outer surface layer of the skin, which can be judged from steady exclusion of the characteristic odor pertaining to toxins, upon application of most of compositions studied so far. Furthermore, the fullerene containing cosmetics of the present invention are bactericides and protect the skin from bacterial attack. The fullerenes clusters and fullerene herein are artificial molecules to which the bacteria have no natural defenses. Bacteria are free living organisms equipped with enzymes to break down and assimilate a wide variety of molecules. However, without wishing to be bound, it is believed that bacteria cannot assimilate the fullerene molecule, while the latter is deemed capable of blocking enzyme activity by restricting conformational movements in enzyme macromolecule, thus eventually causing the bacteria demise.

The fullerene cluster of the present invention exhibits anti-inflammatory and antiseptic activity. They have reparative activity for wounds, as defined herein, and decrease water accumulation under the skin and water swelling of tissue. They have an anti-genesis effect and retard granulated tissue when the tissue is repaired and retard the growth of epithelial layer of skin excess.

More specifically, the fullerene composition comprising fullerene clusters is a beneficial agent for the enhancement or improvement of the healing of wounds in soft tissues (i.e. non-mineralized tissues) such as collagen or epithelium containing tissues, including skin and mucosa, muscle, blood and lymph vessels, nerve tissues, glands, tendons, eyes and cartilage. It exerts especially useful effects in the healing or prophylaxis of soft tissue wounds.

Accordingly, the present invention relates to the use of fullerene clusters in a pharmaceutical and/or cosmetic composition i) for healing of a wound, ii) for improving healing of a wound, and/or iii) for soft tissue regeneration and/or repair.

In another aspect, the invention relates to a method of improving the healing of a wound or of promoting soft tissue regeneration and/or repair, the method comprising administering, to a mammal in need thereof, a therapeutically or prophylactically effective amount of fullerene clusters.

Further, the fullerene clusters have anti-bacterial and/or anti-inflammatory properties that can be used for treatment of both soft and hard (i.e. mineralized) tissue conditions.

As used herein, wounds and/or ulcers are normally found protruding from the skin or on a mucosal surface or as a result of an infarction in an organ (“stroke”). A wound may be a result of a soft tissue defect or a lesion or of an underlying condition. In the present context the term “skin” relates to the outermost surface of the body of an animal including a human and embraces intact or almost intact skin as well as an injured skin surface. The term “mucosa” relates to undamaged or damaged mucosa of an animal such as a human and may be the oral, buccal, aural, nasal, lung, eye, gastrointestinal, vaginal, or rectal mucosa.

In the present context the term “wound” denotes a bodily injury with disruption of the normal integrity of tissue structures. The term is also intended to encompass the terms “sore”, “lesion”, “necrosis” and “ulcer”. Normally, the term “sore” is a popular term for almost any lesion of the skin or mucous membranes and the term “ulcer” is a local defect, or excavation, of the surface of an organ or tissue, which is produced by the sloughing of necrotic tissue. Lesion generally relates to any tissue defect. Necrosis is related to dead tissue resulting from infection, injury, inflammation or infarctions.

The term “wound” used in the present context denotes any wound and at any particular stage in the healing process including the stage before any healing has initiated or even before a specific wound like a surgical incision is made (prophylactic treatment).

Examples of wounds which can be prevented and/or treated in accordance with the present invention are, e.g., aseptic wounds, contused wounds, incised wounds, lacerated wounds, non-penetrating wounds (i.e. wounds in which there is no disruption of the skin but there is injury to underlying structures), open wounds, penetrating wounds, perforating wounds, puncture wounds, septic wounds, subcutaneous wounds, and the like. Examples of sores which can be prevented and/or treated in accordance with the present invention include bed sores, canker sores, chrome sores, cold sores, pressure sores, and the like. Examples of ulcers which can be prevented and/or treated in accordance with the present invention include, e.g., peptic ulcer, duodenal ulcer, gastric ulcer, gouty ulcer, diabetic ulcer, hypertensive ischemic ulcer, stasis ulcer, ulcus cruris (venous ulcer), sublingual ulcer, submucous ulcer, symptomatic ulcer, trophic ulcer, tropical ulcer, veneral ulcer, e.g. caused by gonorrhoea (including urethritis, endocervicitis and proctitis). Conditions related to wounds or sores which may be successfully treated according to the invention are burns, anthrax, tetanus, gas gangrene, scalatina, erysipelas, sycosis barbae, folliculitis, impetigo contagiosa, or impetigo bullosa, and the like. There is often a certain overlap between the use of the terms “wound” and “ulcer” and “wound” and “sore” and, furthermore, the terms are often used at random. Therefore as mentioned above, in the present context the term “wounds” encompasses the term “ulcer”, “lesion”, “sore” and “infarction”, and the terms are indiscriminately used unless otherwise indicated.

The kinds of wounds to be treated according to the invention include also i) general wounds such as, e.g., surgical, traumatic, infectious, ischemic, thermal, chemical and bullous wounds; and ii) wounds on the skin such as, e.g., neoplasm, burns (e.g. chemical, thermal), lesions (bacterial, viral, autoimmunological), bites and surgical incisions. Another way of classifying wounds is as i) small tissue loss due to surgical incisions, minor abrasions and minor bites, or as ii) significant tissue loss. The latter group includes ischemic ulcers, pressure sores, fistulae, lacerations, severe bites, thermal burns and donor site wounds (in soft and hard tissues) and infarctions.

In other aspects of the invention, the wound to be prevented and/or treated is selected from the group consisting of aseptic wounds, infarctions, contused wounds, incised wounds, lacerated wounds, non-penetrating wounds, open wounds, penetrating wounds, perforating wounds, puncture wounds, septic wounds and subcutaneous wounds.

Other wounds which are of importance which can be treated or prevented by the fullerene cluster in connection with the present invention are wounds like ischemic ulcers, pressure sores, fistulae, severe bites, thermal burns and donor site wounds.

Ischemic ulcers and pressure sores are wounds which normally only heal very slowly and especially in such cases an improved and more rapid healing is of course of great importance for the patient. Furthermore, the costs involved in the treatment of patients suffering from such wounds are markedly reduced when the healing is improved and takes place more rapidly.

Donor site wounds are wounds which e.g. occur in connection with removal of hard tissue from one part of the body to another part of the body e.g. in connection with transplantation. The wounds resulting from such operations are very painful and an improved healing is therefore most valuable. The fullerene clusters or compositions containing same of the present invention are useful in treating such wounds.

The term “skin” is used in a very broad sense embracing the epidermal layer of the skin and—in those cases where the skin surface is more or less injured—also the dermal layer of the skin. Apart from the stratum corneum, the epidermal layer of the skin is the outer (epithelial) layer and the deeper connective tissue layer of the skin is called the dermis.

Since the skin is the most exposed part of the body, it is particularly susceptible to various kinds of injuries such as, e.g., ruptures, cuts, abrasions, burns and frostbites or injuries arising from various diseases. Furthermore, much skin is often destroyed in accidents. However, due to the important barrier and physiologic function of the skin, the integrity of the skin is important to the well-being of the individual, and any breach or rupture represents a threat that must be met by the body in order to protect its continued existence.

Apart from injuries on the skin, injuries may also be present in all kinds of tissues (i.e. soft and hard tissues). Injuries on soft tissues including mucosal membranes and/or skin are especially relevant in connection with the present invention. The present fullerene clusters or compositions containing same are useful in treating the various wounds, sores, and ulcers described herein.

Healing of a wound on the skin or on a mucosal membrane undergoes a series of stages that results either in repair or regeneration of the skin or mucosal membrane. In recent years, regeneration and repair have been distinguished as the two types of healing that may occur. Regeneration may be defined as a biological process whereby the architecture and function of lost tissue are completely renewed. Repair, on the other hand, is a biological process whereby continuity of disrupted tissue is restored by new tissues which do not replicate the structure and function of the lost ones.

The majority of wounds heal through repair, meaning that the new tissue formed is structurally and chemically unlike the original tissue (scar tissue). In the early stage of the tissue repair, one process which is almost always involved is the formation of a transient connective tissue in the area of tissue injury. This process starts by formation of a new extracellular collagen matrix by fibroblasts. This new extracellular collagen matrix is then the support for a connective tissue during the final healing process. The final healing is, in most tissues, a scar formation containing connective tissue. In tissues which have regenerative properties, such as, e.g., skin and bone, the final healing includes regeneration of the original tissue. This regenerated tissue has frequently also some scar characteristics, e.g. a thickening of a healed bone fracture.

Under normal circumstances, the body provides mechanisms for healing injured skin or mucosa in order to restore the integrity of the skin barrier or the mucosa. The repair process for even minor ruptures or wounds may take a period of time extending from hours and days to weeks. However, in ulceration, the healing can be very slow and the wound may persist for an extended period of time, i.e. months or even years.

The stages of wound healing normally include inflammation (normally 1-3 days), migration (normally 1-6 days), proliferation (normally 3-24 days) and maturation (normally 1-12 months). The healing process is a complex and well orchestrated physiological process that involves migration, proliferation and differentiation of a variety of cell types as well as synthesis of matrix components. The healing process may be separated into the following three phases:
i) Haemostasis and Inflammation

When platelets are present outside the circulatory system and exposed to thrombin and collagen, they become activated and they aggregate. Thus, platelets initiate the repair process by aggregating and forming a temporary plug to ensure haemostasis and prevent invasion from bacteria. The activated platelets initiate the coagulation system and release growth factors like platelet-derived growth factor (PDGF) and epidermal growth factors (EGFs) and transforming growth factors (TGFs).

The first cells to invade the wound area are neutrophils followed by monocytes which are activated by macrophages.

The major role of neutrophils appears to be clearing the wound of or defending the wound against contaminating bacteria and to improve the healing of the wound by removing dead cells and platelets. The infiltration of neutrophils ceases within about the first 48 hours provided that no bacterial contamination is present in the wound. Excess neutrophils are phagocytosed by tissue macrophages recruited from the circulating pool of blood-borne monocytes. Macrophages are believed to be essential for efficient wound healing in that they also are responsible for phagocytosis of pathogenic organisms and a clearing up of tissue debris. Furthermore, they release numerous factors involved in subsequent events of the healing process. The macrophages attract fibroblasts which start the production of collagen.
ii) Granulation Tissue Formation and Re-Epithelization

Within 48 hours after wounding, fibroblasts begin to proliferate and migrate into the wound space from the connective tissue at the wound edge. The fibroblasts produce collagens and glycosaminoglycans and, inter alia, low oxygen tension at the wound stimulates proliferation of endothelial cells. The endothelial cells give rise to the formation of a new capillary network.

Collagenases and plasminogen activators are secreted from keratinocytes. If the wound is left undisturbed and well-nourished with oxygen and nutrients, keratinocytes will migrate over the wound. Keratinocytes are believed only to migrate over viable tissue and, accordingly, the keratinocytes migrate into the area below the dead tissue and the crust of the wound.

The wound area is further decreased by contraction.
iii) Dermal Remodelling

As soon as the re-epithelization is completed the remodelling of the tissue begins. This phase, which lasts for several years, restores the strength to the wounded tissue.

All of the above-mentioned healing-processes take considerable time. The rate of healing is influenced by the wound's freedom from infection, the general health of the individual, presence of foreign bodies, etc. Some pathologic conditions like infection, maceration, dehydration, generally poor health and malnutrition can lead to formation of a chronic ulcer such as, e.g., ischemic ulcers.

Until at least superficial healing has occurred, the wound remains at risk of continued or new infection. Therefore, the quicker the wound can heal, the sooner the risk is removed.

Thus, any procedure that can influence the rate of wound healing or favorably influence the healing of wounds is of great value.

Furthermore, as almost all tissue repair processes include the early connective tissue formation, a stimulation of this and the subsequent processes are contemplated to improve tissue healing.

In the present context the term “clinical healing” is used to denote a situation where no tissue interruption can be visually observed and only discrete signs of inflammation are present such as a light redness or a discretely swollen tissue. In addition, no complaints of pain are present when the organ is relaxed or untouched.

As mentioned above, the invention relates to the use of fullerene clusters, as defined herein as a wound healing agent, i.e. an agent which accelerates, stimulates or promotes healing of dermal or mucosal wounds. Without wishing to be bound it is believed that the fullerene cluster promotes healing of the wound at all of the stages indicated hereinabove. Accordingly, the fullerene clusters of the present invention or composition containing same are useful as tissue regeneration and/or repair agent. Furthermore, due to the wound healing effect, the fullerene clusters of the present invention have pain relief effect.

In a further aspect of the present invention, the fullerene clusters of the present invention are used as therapeutic or prophylactic agents having an anti-microbial effect. They exhibit infection-decreasing properties.

In the present context the term infection-decreasing effect relates to a treating or preventive effect by the fullerene clusters on an infection in a tissue of an individual when the tissue or the individual is treated with the fullerene clusters.

The term “infection” relates to the invasion and multiplication of microorganisms in body tissues or accumulation on the tissues, which may be clinically unapparent or result in local cellular injury due to competitive metabolism, enzymes, toxins, intracellular replication or antigen-antibody response.

In accordance with the present invention, the infection to be prevented and/or treated may be caused by a microorganism. The microorganisms of interest according to the present invention include bacteria, viruses, yeast, molds, protozoa and rickettsiae.

In the present context the term “anti-bacterial effect” means that the growth of bacteria is suppressed or the bacteria are destroyed. The term is not limited to certain bacteria but encompasses in general any bacteria. However, the invention is focused on i) pathogenic bacteria which cause diseases in mammals including humans and/or ii) bacteria which normally are present in a mammal body and which under certain conditions may cause unwanted conditions in the body.

Accordingly, the invention relates to the use of fullerene clusters for the prevention of or treatment of bacterial growth on a body surface such as the skin, a mucosal surface or a nail or a tooth surface.
The fullerene clusters may be used for the treatment of an infection caused by bacteria together with or without the presence of an antimicrobial. Gram negative bacteria to be treated with the fullerene clusters include cocci, such as Neisseria (e.g. N. meningitis, N. gonorrhoeae), and Acinetobacter or rods, such as Bacteroides (e.g. B. fragilis), Bordetella (e.g. B. pertussis, B. parapertussis), Brucella (e.g. B. melitentis, B. abortus Bang, B. suis), Campylobacter (e.g. C. jejuni, C. coli, C. fetus), Citobactor, Enterobacter, Escherichia (e.g. E. coli), Haemophilus (e.g. H. influenzae, H. para-influenzae), Klebsiella (e.g. K. pneumoniae), Legionella (e.g. L. pneumophila), Pasteurella (e.g. P. yersinia, P. multocida), Proteus (e.g. P. mirabilis, P. vulgaris), Pseudomonas (e.g. P. aeruginosa, P. pseudomallei, P. mailer), Salmonella (e.g. S. enteritidis, S. infantitis S. Dublin, S. typhi, S. paratyphi, S. schottmulleri, S. choleraesuis, S. typhimurium, or any of the 2,500 other serotypes), Serratia (e.g. S. marscences, S. liquifaciens), Shigella (e.g. S. sonnei, S. flexneri, S. dysenteriae, S. boydii), Vibrio (e.g. V. cholerae, V. el tor), and Yersinia (e.g. Y. enterocolitica, Y. pseudotuberculosis, Y. pestis). Gram positive bacteria to be treated with the fullerene clusters include cocci, such as Streptococcus (e.g. S. pneumoniae, S. viridans, S. faecalis, S. pyogenes), Staphylococcus (e.g. S. aureus, S. epidermidis, S. saprophyticus, S. albus), and rods, such as Actinomyces (e.g. A. israelli), Bacillus (e.g. B. cereus, B. subtilis, B. anthracis), Clostridium (e.g. C. botulinum, C. tetani, C. perfringens, C. difficile), Corynebacterium (e.g. C. diphtheriae), Listeria, and Providencia. Other bacteria causing infections include Propionobacterium acne and Pityosporon ovale and the like. Diseases or maladies caused by or resulting from infections by these bacteria are also treated by the fullerene clusters or compositions containing same of the present invention.

The fullerene clusters or compositions containing same are also used for the treatment of an infection caused by a spirochete such as, e.g., Borrelia, Leptospira, Treponema or Pseudomonas.

The fullerene clusters can be used in conjunctions with an antimicrobial. An antimicrobial to be used in combination with the fullerene clusters include an antimicrobial that has an antimicrobial action through inhibition of cell wall synthesis, such as beta-lactams and vancomycin, preferably penicillins, such as amdinocillin, ampicillin, amoxicillin, azlocillin, bacampicillin, benzathine pinicillin G, carbenicillin, cloxacillin, cyclacillin, dicloxacillin, methicillin, mezlocillin, nafcillin, oxacillin, penicillin G, penicillin V, piperacillin, and ticarcillin; cephalosporins, such as the first generation drugs cefadroxil, cefazolin, cephalexin, cephalothin, cephapirin; and cephradine, the second generation drugs cefaclor, cefamandole, cefonicid, ceforamide, cefoxitin, and cefuroxime, and the third generation cephalosporins, cefoperazone, cefotaxime, cefotetan, ceftazidime, ceftizoxime, ceftriaxone, and moxalactam; carbapenems such as imipenem; and monobactams such as aztreonam, and the like.

Other antimicrobial drugs which can be used in conjunction with the fullerene clusters of the present invention include those antimicrobials with action through inhibition of protein synthesis, such as chloramphenicol; other tetracyclines, preferably demeclocycline, doxycycline, methacycline, minocycline, and oxytetracycline; aminoglycosides such as amikacin, gentamicin, kanamycin, neomycin, netilmicin, paromomycin, spectinomycin, streptomycin, and tobramycin; polymyxins such as colistin, colistimathate, and polymyxin B, and erythromycins and lincomycins; antimicrobials with action through inhibition of nucleic acid synthesis in particular sulfonamides such as sulfacytine, sulfadiazine, sulfisoxazole, sulfamethoxazole, sulfamethizole, and sulfapyridine; trimethoprim, quinolones, novobiocin, pyrimethamine, and rifampin.

The present invention also relates to the uses of fullerene clusters as therapeutic or prophylactic agents having an anti-inflammatory effect.

Several drugs are employed to suppress the manifestations of inflammation, including the adrenocorticosteroids, the large group comprising the so called non-steroid anti-inflammatory drugs or NSAIDs, and drugs such as immunosuppressive agents. Adrenocorticosteroids, and especially glucocorticoids, have potent anti-inflammatory effects when used in pharmacological doses. They specifically inhibit the early vascular phase of the inflammatory process by decreasing the vascular permeability and thereby granulocyte migration. Glucocorticoids also interfere with late inflammatory and reparative processes, in that they inhibit the proliferation of mesenchymal cells and the production of extracellular macromolecules, including proteoglycanes and collagen. It has been shown experimentally that glucocorticoids inhibit, for example, macrophage function, production of humoral. antibodies, cellular immunity, and possibly the release of lysosomal enzymes. These drugs may be utilized in conjunction with the fullerene clusters of the present invention.

The severity of tissue damage may depend on the antigen/antibody reaction of the organism as well as the degree of retention of inflammatory products in the affected area. Accumulation of mediators of local inflammation accelerates the process. In most cases the process is slow, with immunoinfiltration of the tissue and formation of granulation tissue which contains inflammatory cells.

In the present context the term “anti-inflammatory effect” denotes a counteracting or suppression of inflammation.

The inflammatory condition to be treated in accordance with the present invention may of course be any inflammatory condition in/on any part of the body or any inflammatory condition present in soft or hard tissue.

In the treatment of wounds, and in the treatment and/or prophylaxis of inflammation, infections and the like, the effective amount of fullerene clusters can be determined easily by a physician. The amounts will vary depending on the health and age of the mammal being treated, the condition being treated, the severity of the condition being treated, and the like. A physician will be able to determine the amounts without undue experimentation. A preferred amount is the same as described herein with respect to the cosmetic compositions. Moreover, the pharmaceutical compositions may contain the same components as described hereinabove for the cosmetic compositions.

The cosmetic compositions and the pharmaceutical compositions of the present invention may be exposed to light. In fact, the present composition may be more effective when exposed to light. This is especially true for the pharmaceutical composition of the present invention comprising an effective amount of fullerene clusters especially those used for wound healing. The pharmaceutical activity of the compositions of the present invention are enhanced in normal solar or artificial light. Preferably, the light ranges from about 20 to about 100 LX light intensity. Nevertheless, sufficient light is obtained in normal everyday life, such as from sunlight as well as less intense light, such as that found in a room from an electric bulb or even fluorescent light, to effectuate the therapeutic activity, such as wound healing, of the fullerene clusters in the present compositions.

The following non-limiting examples of the production of fullerene clusters and their use in a brand line of cosmetic products additionally illustrate the present invention.

### EXAMPLE 1

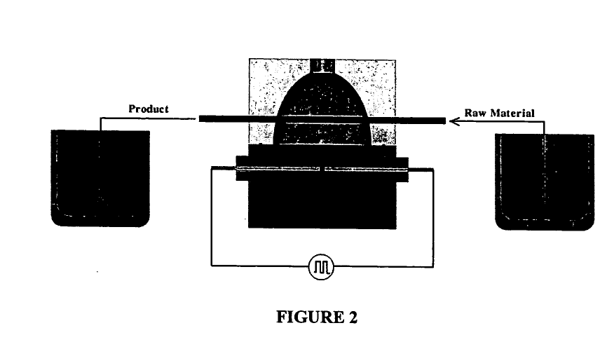

The solvent free fullerite C₆₀ of 99.9 wt. % purity is obtained by subliming the crystalline solvate of C₆₀ in vacuum, then grinding it in an agate mortar in an argon dry box to the average crystallite size of about 10 microns. 400 mg of this powder is placed into a thin-walled polyethylene test tube, which is thereafter filled with deionized (DI) water to capacity (3 ml) and hot sealed at the open end. The sample thus prepared is inserted through the orifice in the upper lid of the electrohydraulic apparatus (FIG. 2) into the chamber filled with DI water that is circulating through a cooler. The sample is secured with a special holder so that the fullerite powder is located in the upper focus of the ellipsoid. A series of electric discharges with 2 Hz pulse frequency is effected in the lower focus of the ellipsoid for a total of two hours with a 5 μF capacitor which is charged between the pulses to 6,000 V with a high voltage power supply. The apparatus produces about 75 J energy per pulse, and the pulse front rise time is about 30 ns with total pulse duration about 500 ns. Upon completion of EH treatment the tube is unsealed and a droplet of appropriately thinned solution of the product is dried and gold-coated in argon plasma to prepare the sample for high-resolution SEM inspection. The SEM study reveals the presence of small fullerene clusters sized down to 7 nm in diameter, with the average diameter about 15 nm. An aliquot of the fullerene cluster product is ultrasonically dispersed in a large amount of DI water to form a solution with fullerene concentration about 0.1 mg/ml. This solution is filtered through the 0.22 μm porous PTFE filter to yield transparent yellow-brown solution of fullerene clusters that is stable in the dark at 5 C for at least 5 months. The rest of the fullerene cluster product is dried in air at room temperature and stored in a refrigerator before its use in preparing cosmetic compositions. It can be re-dispersed in water, alcohol or oils by soft ultrasonic treatment to yield stable yellow to dark-brown transparent solutions of fullerene clusters, depending on type of solvent and fullerene concentration, which may vary from 0.1 to 40 mg/ml in different liquids used. The thus obtained fullerene clusters can be used in various cosmetic compositions, in accordance with the present invention.

### EXAMPLE 2

The same EH treatment as in Example 1 was applied to a suspension of 400 mg of 99.9 wt.% pure fullerite C₆₀ in 3 ml of clear olive oil, except that total duration of the treatment was 4 hours. Several interruptions of the treatment were necessary to adjust the clearance between electrodes, which grows because of the electrode's spark erosion. High-resolution SEM inspection revealed the presence of fullerene clusters with an average diameter of about 20 nm in a sample prepared by 1:10,000 dilution of the as-produced product in ethanol with subsequent drying of a droplet of the ethanol solution of a polished aluminum support. The as-produced product was stored in a refrigerator and dissolved easily under soft ultrasonic treatment in a larger amount of olive or other natural oil of interest for cosmetics.

### EXAMPLE 3

A slurry of 100 mg of finely ground 99.9% pure fullerite C₆₀ in 200 ml of reagent grade ethanol, maintained and suspended by a magnetic stirrer in a two-neck glass flask, was further circulated by a peristaltic pump at a rate of 20 ml/min in a closed contour containing a thin PTFE tube passing through the upper focal point of the ellipsoidal chamber of FIG. 2. The conditions of the EH treatment are the same as in EXAMPLE 1 except that duration of treatment was 4 hours. After filtering the product through a 0.22 μm PTFE filter, a slightly opalescent reddish solution was obtained with a fullerene concentration of about 0.25 mg/ml. The SEM study established abundant fullerene clusters in the size range 7 to 30 nm and larger clusters 50 to 150 nm in diameter which amounted to about one third of the sample mass. An aliquot of the product was stored in a refrigerator without signs of precipitation for at least 5 months. Additional prolonged ultrasonic treatment of the sample did not remove larger clusters. The product was concentrated and dried without losing the ability to re-dissolve in ethanol, water and natural oils for further use in cosmetics.

### EXAMPLE 4

Mixed fullerite containing C₆₀ and C₇₀ in a 5:1 molar ratio (100 mg) was dissolved in dried and distilled toluene (100 ml) and added to the solution of 10 g PVP (MW 10,000-15,000) in dried and distilled chloroform (200 ml). Stirring of the combined solution was performed for at least 6 hours until the mixture became brown, then the solvent was removed by vacuum drying. The solid product was mixed with water (150 ml) and subjected to 20 kHz ultrasonic treatment at a power density 40 W/cm³ for 3 min to yield a brown solution, which was clearly distinct in color from the orange solution of mixed fullerite. The solution was filtered through a dense glass filter, then the solvent was removed with a rotavapor at 50 C and the product dried in vacuum at 50 C.

The light-brown dry powder obtained was a water-soluble form of fullerene clusters that was used subsequently in preparing the cosmetic cream compositions. The samples of these fullerene creams, incorporating such transport facilitating ingredients as retinal, alpha-hydroxy acids and antioxidants at a concentration amounting to about 5 wt. % of the cream, have perfect homogeneous consistency, are easy to spread and are readily absorbed by different skin types.

### EXAMPLE 5

The following composition was prepared:
Animal fat: 40-80%
Active fullerene cluster component prepared in EXAMPLE 1: 0.05-0.25%
Ethyl alcohol: 20-60%
Glycerine: 0-30%
Polyethylene glycol (PEG) and/or polypropylene glycol (PPG) and/or polyvinylpyrrolidone (PVP) 0.0001-3%
Extracts of simples (by weight of solid extracted substance) and/or bees propolis and/or other apiculture products and/or mumie and/or birch tar: 0.0001-25%
Surfactant: 0.0001-0.5%
Dye: 0.0001-0.5%
Fragrance: 0.0001-0.5%
Water: the rest to 100%

The components were mixed together and formulated into an ointment.

A panel of volunteers consisting of 10 females evaluated the above product with 0.25% content of active fullerene compound and a control product that lacked the compound, but was otherwise identical, in a single use test. The products were applied randomly to the skin of the left and right hands and were worn under normal conditions for 5 hours, then evaluated for personal feelings and appearance of the skin. The overall opinion of the product performance was judged in terms of skin freshness, shine, color, smoothness, and personal feelings of pleasure and lightness of wear. In addition, color photographs of hands were taken, and images were assessed by expert graders of skin state in blind testing. The fullerene product was judged superior to the control product, based on panelist responses and expert visual assessment.

### EXAMPLE 6

The following composition was prepared:
Glycerine and/or polyethylene glycol and/or polypropylene glycol and/or polyvinylpyrrolidone and/or their copolymers, and also copolymers of the listed classes of polymers containing grafted biologically active function groups: 2-30%
Vegetable oil and/or animal oil and/or mammal fat, birds, fishes, amphibians, reptiles, arthropoda: 0.0001-5.0%
Mineral and/or synthetic oil (silicone, fluorosilicone, siloxane, etc.) 0.0001-5.0%
Active fullerene cluster component prepared in EXAMPLE 4: 0.05-0.25%
Ethyl alcohol: 2.5%
Extracts of simples (by weight of solid extracted substance) and/or bees propolis and/or other apiculture products and/or mumie and/or birch tar: 0.0001-25%
Surfactant 0.0001-0.5%
Dye: 0.0001-0.5%
Fragrance: 0.0001-0.5%
Starch, gelatin or other thickener: 0.5%
Water: to 100%

The components were mixed together and formulated into a cream.

### EXAMPLE 7

A composition containing 0.05% active fullerene cluster component prepared as described in EXAMPLE 4 and mixed with a vaseline base was used to quantitatively study its reparative activity, when the cream was topically applied to wounded animal skin. The skin of three animal groups, each consisting of ten 180-200 g while rats was subjected to a specific treatment to cause formation of 2.0 by 2.0 cm conventionally aseptic wounds. The first group was then treated with the composition containing active fullerene component on the wound, and the control group with pure Vaseline. The third group was treated with an effective antiseptic, 10% methyluracyl ointment for comparison. The wound reparation experiment was carried out at illumination intensity of 200 Lux. The remedy efficiency was judged by the rate of wound area contraction, the term of total recovery and by assessing the state of wound by hystomorphological studies. The composition with active fullerene component led to the reduction of the average wound area of 45% and 23% of the original value on the 7th and 14th day, respectively with total coverage of the wound area with epithelium, that is, total recovery of the wound on the 14th day. In the control group, obtaining baseline the appropriate changes were 85%, 73% and 65% of the original size on the 7th, 14th and 21st day, respectively, thus showing much slower wound closing rate in the absence of fullerene component. With total recovery attained on the 28th day. The 10% methyluracyl ointment showed 50%, 30% and 12% of the original wound area on the 7th, 14th and 21st day, and total recovery on the 21st day. Thus, the 0.05 fullerene cluster composition reparative activity is quite comparable with that of 10% methyluracyl ointment, and in particular, fullerene clusters are distinctly superior in stimulation of the rate of epithelization.

Similar studies performed at illumination intensity of only 5 Lux (in the dark) have revealed much smaller differences between experimental and control groups. This result evidences the photodynamic activity of the fullerene component and its important role in achieving a reparative effect. Without wishing to be bound, it is believed that the role of photodynamic activity in this case probably was caused by the formation of singlet oxygen, that stimulates intracellular reparative processes by causing a minor primary damages to biological membranes.

### EXAMPLE 8

The following composition was prepared:
Vegetable oil and/or animal oil and/or fat of mammals, birds, fishes, amphibians, reptiles, arthropoda. (Examples: fat of pork, badger, bear, dog, seal, crocodile, ostrich): 40-80%
Active fullerene cluster component prepared in EXAMPLE 2: 0.05-0.25%
Stearine and/or lanoline and/or spermaceti: 0.0001-30.0%

Extracts of simples (medicine plants). Examples include: extracts of juniper needles and berries, stone-pine, plantain, milfoil, wild rosemary, St.-John's wort, parsley, celery, roots of tormentil, leaf-buds of birch and black currant, shell of walnuts and cedar nuts, fruits of henbane, fruit body of fly-agarics (amount by weight of solid extracted substance) and/or bees propolis and/or other apiculture products and/or mumie and/or birch tar: 0.0001-20%
Surfactant 0.0001-0.5%
Dye: 0.0001-0.5%
Fragrance: 0.0001-0.5%
Disperse mineral substances, with particle size permitted for the medical purposes, such as ZnO, TiO₂, MgSO₄, BaSO₄, chalk, talc, mineral pigments: 0.0-10%
Water: the rest

The components were mixed together and formulated into an ointment.

### EXAMPLE 9

A panel of volunteers consisting of 6 females and 6 males was used to evaluate, in a single use test, a composition such as is described in Example 8 (containing 0.25% w/w of active fullerene compound) against a control product that lacked the compound, but was otherwise identical. The composition and control were applied to the skin of the left and right cheeks of each volunteer either directly or in reverse order, according to a deterministic pseudo random code with the same statistical properties as a random distribution. The composition and control were worn outdoors under cold, windy weather conditions (5° C.) with moderate midday sunshine for 3 hours. After a half an hour at room temperature, for the full skin sensitivity to return to each cheek, the composition and control were evaluated for the volunteers' subjective sensations and self evaluation of skin appearance. The overall opinion of the product performance was judged in terms of skin freshness, shine, color, smoothness, and expressed comments of favorable or unfavorable. In addition, color photographs of the left and right cheeks were taken, and expert graders of skin state assessed these images. The composition was more frequently rated as protective by the volunteers. The fullerene product was judged superior to the control product in respect of protecting the skin against severe weather conditions, based on panelist responses and expert visual assessment.

### EXAMPLE 10

The following composition was prepared:
Active fullerene cluster components prepared in EXAMPLE 3: 0.05-0.25%
Ethyl alcohol: 40-50%
Extracts of simples (by weight of solid extracted substance) and/or bees propolis and/or other apiculture products and/or mumie: 0.0001-15%
Fragrance of a natural vegetative origin: 0.0001-0.5%
Double-distilled water: the rest
The components were mixed and made into a perfume.

### EXAMPLE 11

The following composition was prepared as a moisturizing cream from the following:
Stearine cosmetic 1.5%
Wax emulsion 2.0%
Stearate PEG 400 0.5%
Perfume oil 4.0%
Glycerol distilled 7.0%
Vitamin F 3.0%
Triethanolamine 0.4%
Sodium laurylsulfate ethoxylated (EMAL 270 D) 0.5%
Ethanol distilled 2.0%
Oil extract of nettle 0.5%
Oil extract of chamomile 0.5%
Oil extract of mint 0.5%
Oil extract of sweetbrier 0.5%
0.05% solution of fullerene clusters in Sea Blacksoarn oil 5.0%
Paraoxybenzoic acid ethyl ester 0.3%
Paraoxybenzoic acid propyl ester 0.2%
Fragrance 0.3%
Water distilled—to 100%

The various components were mixed together and formulated into a moisturizing cream.

The above composition meets the cosmetic industry standards for moisturizing creams. It is a viscous, cream-like, light beige to beige homogeneous mass which does not contain particulate matter or impurities and which has a pleasant herbal fragrance and good user properties. Preliminary comparative testing of this cream relative to a control containing all of the above ingredients except the fullerene clusters on volunteers suggests additional beneficial properties from incorporation of an active fullerene component, including enhancement of anti-aging and anti-wrinkle effects, resulting in well-expressed rejuvenation of the skin.

### EXAMPLE 12

The following composition was prepared as a softening cream from the following components:
Glyceryl stearate-PEG-759 stearate (CELOT 64) 3.5%
Mineral oil 5.0%
Cetyl stearyl alcohol 2.0%
Dimethicone 5.0%
Glycerine distilled 5.0%
Wheat germ oil 3.0%
0.05% solution of fullerene clusters in Sea Blacksoarn oil 5.0%
Triethanolamine 0.15%
Carbopol 980 (ETD 2001) 0.2%
Conservant Euksyl K 100 (benzyl alcohol, methylchloroisothiazolinone, methylisothiazolinone 0.1%
Fragrance 0.3%
Water distilled—to 100%

These components were mixed together and formulated into a softening cream.

This composition meets the cosmetic industry standards for softening creams. It represents a viscous, cream-like, light beige homogeneous mass which does not contain particulate matter or impurities and has good user properties. Preliminary comparative testing of this cream relative to a cream containing all of the components listed hereinabove except the fullerene clusters on volunteers suggests additional beneficial properties attributable to incorporation of an active fullerene component, including rejuvenation of the skin.

### EXAMPLE 13

The following composition was prepared as a cream from the following components:
TEFOSE 2561 (PEG-6 stearate cetet-20, glycerylstearate stearate 20) 5.0%
Mineral oil 3.0%
Cetyl stearyl alcohol 3.0%
Dimethicone 3.0%
Glycerine distilled 4.0%
0.05% solution of fullerene clusters in linolic acid 1.7%
Vitamin F in oil 2.0%
Wine extract (Optivegetol wine P-150 hydro) 4.0%
Ginseng extract in aqueous alcohol 5.0%
Triethanolamine 0.2%
Carbopol 980 (ETD 2001) 0.15%
Conservant Euksyl K 100 (benzyl alcohol, methylchloroisothiazolinone, methylisothiazolinone 0.1%
Fragrance 0.3%
Water distilled—to 100%

The various components were mixed together and formulated into an anti-aging cream.

The above composition meets the cosmetic industry standards for anti-age creams. It is a viscous, cream-like, white light beige homogeneous mass that does not contain particulate matter or impurities. Preliminary testing of this cream relative to a cream containing all of the above ingredient except the fullerene clusters on volunteers suggests a pronounced ant-age effect due to incorporation of an active fullerene component.

### EXAMPLE 14

The following composition was prepared from the following components as a gel:
Carbopol 980 0.4%
Glycerine distilled 4.0%
Dimethicone 1.0%
0.05% solution of fullerene clusters in linolic acid 1.7%
Glucose (mannite) 0.5%
Wine extract (Optivegetol wine P-150 hydro) 5.0%
Thiourea (carbamide) 1.0%
Triethanolamine 0.4%
Green dye mixture (E 102, E 133) p.s.
Conservant Euksyl K 100 (benzyl alcohol, methylchloroisothiazolinone, methylisothiazolinone 0.1%
Fragrance 0.3%
Water distilled—to 100%

The above components were mixed together and formulated into an anti-aging gel.

This composition meets the cosmetic industry standards for anti-age gels. It is a gel-like, light green homogeneous mass that does not contain particulate matter or impurities and which has a pleasant fragrance. Preliminary comparative testing of the gel relative to a gel comprised of all of the above ingredients except the fullerene clusters on volunteers suggests a distinct anti-age effect due to incorporation of an active fullerene component.

### EXAMPLE 15

The following composition was prepared from the following components ad an anti-inflammatory cream:
Glyceryl stearate PEG-75-stearate (CELOT 64, non-ionogenic detergent) 3.5%
Mineral oil 5.0%
Cetyl stearyl alcohol 2.0%
Dimethicone 5.0%
Glycerine distilled 5.0%
Oats extract 2.0%
Oil extract of marigold 1.0%
Oil extract of thyme 1.0%
Oil extract of chamomile 1.0%
Oil extract of liquorice 1.0%
0.05% fullerene cluster content PVP/fullerene complex 5.0%
Triethanolamine 0.15%
Carbopol 980 (ETD 2001) 0.2%
Conservant Euksyl K 100 (benzyl alcohol, methylchloroisothiazolinone, methylisothiazolinone 0.1%
Water distilled—to 100%

The above components were mixed together and formulated into an anti-inflammatory cream.

This composition meets the cosmetic industry standards for anti-inflammatory creams. It is a viscous, cream-like, light beige homogeneous mass that does not contain particulate matter or impurities and which has a pleasant herbal fragrance. Preliminary comparative testing of this cream relative to a cream containing the same components as above except fullerene clusters on volunteers suggests an enhanced anti-inflammatory effect due to incorporation of an active fullerene component. The same cream composition, without the PVP/fullerene component but with 0.3% ultradispersed fullerite produced as in EXAMPLE 1, which is a fullerene cluster product, produces a similarly strong anti-inflammatory effect.

### EXAMPLE 16

The following composition was prepared as moisturizing vanishing cream.
Fullerene C60 in cluster form 0.05%
White petrolatum USP (Source: Ultima White Petrolatum, PENRECO) 0.8%
Lanolin Alcohol, NF (Super Hartolan, CRODA) 1.0%
PPG-2-Myristyl ether propionate (Crodamol PMP, CRODA) 4.2%
Mineral Oil (Light), NF (Drakeol 7, Light, PENRECO) 8.3%
Triethanolamine, USP, 98% (SIGMA CHEMICAL CO.) 0.6%
Paragon III (McINTYRE) 0.6%
Glycerol distilled, USP (RITA) 3.3%
Stearic Acid, USP (Ritastearic, RITA) 16.7%
Isopropyl Palmitate (Estol 1517, UNIQUEMA) 12.5%
Fragrance: a 50:50 mixture of the fragrances Ocean and African Rain 0.2%
Water distilled, USP—to 100%
Manufacturing Procedure (100 g Batch):

White Petrolatum, Lanolin Alcohol, and Stearic acid were mixed together with 4 g of Mineral Oil (Light) in a 250 mL beaker to form a mixture, and then the mixture was melted by heating it to 60C utilizing a hot plate/stirrer. As soon as the mixture melted, the contents of the beaker were stirred using a Teflon coated spinbar.

Separately PPG-2-myristyl ether propionate and Isopropyl Palmitate were mixed together to form a second mixture in a separate 75 mL beaker and this mixture was heated to 60 C. When the temperature of the second mixture reached 60° C., the PPG-2-myristyl ether propionate and Isopropyl Palmitate were added to the first mixture stepwise. 50 mg Of C₆₀ powder was macerated with 4.3 g of Mineral Oil, then subjected to ultrasonic treatment (20 kHz, 20 W for 30 min) and preheated to 60 C. When the temperature of this mixture reached 60° C., it was added to the mixture containing PPG-2-myristyl ether propinate, Isopropyl plamitate, white petrolatum lanolin alcohol, and stearic acid and mineral oil. The resulting mixture was maintained at 60 C for 20 min while continuing stirring. It was noted that it was important not to overheat the mixture as some charring of the oils may occur which will manifest itself as a change in the color of the solution from a bright clear purple to a more opaque brownish colored solution.

In a separate container, Triethanolamine (base), Glycerin (emollient), Paragon III (preservative) and all of the water were mixed and this mixture was preheated to 60 C.

The oil phase components were added to the aqueous phase components and vigorously stirred (300 rpm, Silverson share mixer) until the mixture congealed and was homogenous; then the stirring was increased to 1000 rpm and maintained for another 20 minutes. Then the heating was discontinued and the mixture was allowed to cool. When the mixture was at 40° C. or below, the fragrance was added thereto and the mixture was mixed at 300 rpm for 2 min.

The resulting mixture was placed in a labeled, opaque container and allow to sit for 24 hours to allow pearlescence to occur, which is due to in-situ soap formation and crystallization.

The C₆₀ Pearlescent cream obtained from the above procedures provided a high degree of emolliency and an antioxidant potency. The emollients present in the formulation (White Petrolatum, Mineral Oil, Glycerin, Isopropyl Palmitate) are designed to form an occlusive barrier on the skin and help prevent the loss of moisture, which in turn enhances the skin's appearance by decreasing environmental and age-related damage that can lead to skin wrinkling, discoloration, and loss of elasticity. In addition, the vanishing cream formula utilized (inclusion of PPG-2-Myristyl ether propionate and Isopropyl Palmitate) permits rapid blending of the formulation into the skin without the greasy feel often associated with occlusive oil-based formulations.

This composition meets the cosmetic industry standards for moisturizing creams. It is a viscous, cream-like, light purple homogeneous mass which does not contain particulate matter or impurities and which has a pleasant fragrance. Preliminary testing of the cream on volunteers suggests additional beneficial properties attributable to incorporation of an active fullerene component, including enhancement of anti-aging and anti-wrinkle effects.

### EXAMPLE 17

Quantitative assessment of the antioxidant activity of fullerene containing compositions.

The quantitative assessment of the antioxidant activity of the cosmetic composition containing fullerene clusters was tested utilizing bromine-based oxidants generated electrochemically.

Procedure:
1. Electrogeneration of bromine-based oxidants and coulonometric tritration were performed with a potentiostate using 1 cm² platinum working electrode at a current 5 mA
2. Bromine oxidant was generated 0.2 M KBr dissolved in 0.1M H₂SO₄/tartrate buffer solution, which ensure 100% yield of oxidant by current. The final point was determined amperometrically with two polarized platinum electrodes at E=20 mV.
3. Samples for analysis in water or water/alcohol media were obtained by maceration of the mixture of a cosmetic containing the active ingredient listed in the following table and this media taken in proporation 1:5 by weight.
4. Antioxidant activity (AOA) is expressed as amount of Coulombs spent for titration of 100 g of the analyzed starting cosmetic substance.

Results:

Bromine compounds including Br₂, Br₃⁻ and active bromo-radicals are electrochemically generated and adsorbed at the surface of the platinum electrode and then these radicals act as oxidating agents in redox reactions, reactions of electrophilic substitution and addition to double bonds e.g. in the fullerene molecules in the fullerene cluster of cosmetic composition. The AOA of a composition containing fullerene cluster was compared with a control comprised of a composition which was fullerene-free, but otherwise identical to the fullerene-containing sample. The measured AOA values are presented in the following Table. For comparison, the AOA of frequency used in cosmetics extracts of medicinal plants are also presented in the following table.

| Analyzed material | AOA, kCb/100 g |
|---|---|
| 0.5 wt % C₆₀/PVP complex | 240.0 |
| PVP without C₆₀ | 21.5 |
| 0.5 wt. % C₆₀ cluster solubilized in | 210.0 |
| Blacksoarn oil | |
| Blacksoarn oil without C₆₀ | 12.3 |
| 0.5 wt % C₆₀ cluster solubilized in Linoleic | 202.4 |
| Acid | |
| Linoleic Acid without C₆₀ | 4.8 |
| 0.5 wt % C₆₀ cluster solubilized in | 180.2 |
| Rapeseed Oil | |
| Rapeseed Oil without C₆₀ | 3.2 |
| Basil herb leaves (Saturated water extract) | 16.5 |
| Meadow-sweet herb leaves (Saturated | 41.0 |
| water extract) | |
| Peppermint herb leaves (Saturated water | 8.5 |
| extract) | |
| Peppermint Birch tree buds (Saturated | 10.0 |
| water/ethanol extract) | |
| Rodiola Rosea root (Saturated | 4.1 |
| water/ethanol extract) | |
| Valerian herb leaves (Saturated | .48 |
| water/ethanol extract) | |
| Hawthorn Berry (Saturated water/ethanol | 0.36 |
| extract | |

As shown by the data in the table, specific AOA of C₆₀ (fullerene cluster) far surpasses that of water and water/ethanol extracts of medicinal plants and of natural oils. This clearly demonstrates the ability of fullerenes to catalytically scavenge and recombine free radicals in solution. In these processes the fullerene molecule is not sacrificed, contrary to what is happening to other known antioxidants. One fullerene molecule can be used several times to deactivate, by rough estimation at least thousands of free radicals.

Unless indicated to the contrary, all percentages are weight percentages. Moreover, the singular shall refer to plural and vice versa.

The above-preferred embodiments and examples were given to illustrate the scope and spirit of the present invention. These embodiments and examples will make apparent to those skilled in the art other embodiments and examples. These other embodiments and examples are within the contemplation of the present invention. Therefore, the present invention should be limited only by the appended claims.

1. A cosmetic composition which is comprised of a cosmetically effective amount of fullerene clusters in association with a cosmetically acceptable carrier.

2. The cosmetic composition according to claim 1 wherein the fullerene clusters are present in an amount effective to prevent or retard free radical and molecular oxidation processes in the skin.

3. The cosmetic composition according to claim 1 wherein the fullerene clusters are associated with a water soluble polymer or porphyrin or a combination thereof.

4. The cosmetic composition according to claim 3 wherein the water soluble polymer is polyvinyl pyrrolidone, polyethylene glycol, polypropylene glycol, copolymers of vinyl pyrrolidine, ethylene glycol or propylene glycol.

5. The cosmetic composition according to claim 1 wherein the fullerene clusters are present in an amount ranging from 0.01 to about 50% by weight relative to the total weight of the composition.

6. The cosmetic composition according to claim 5 wherein the fullerene cluster is present in an amount ranging from about 0.02% to about 10% by weight of the composition.

7. The cosmetic composition according to claim 6 wherein the fullerene cluster is present in an amount ranging from about 0.25% to about 2% by weight of the composition.

8. The cosmetic composition according to claim 1 which additionally comprises a mineral oil, animal oil, or vegetable oil or synthetic oil.

9. The cosmetic composition according to claim 1 which additionally comprises at least one of the following: a film-forming compound, an emulsifier, a thickening agent, a lubricant, a coloring agent, a moisturizing agent, an anti-oxidant, an emollient, a pH adjusting agent, a preservative, a sunscreen agent, a buffer, perfume, an additive preventing perspiration or an additive repelling sanguivorous insects or bactericides.

10. The cosmetic composition according to claim 1 comprised of one type of fullerene.

11. The cosmetic composition of claim 10 wherein the fullerene is C₆₀.

12. The cosmetic composition according to claim 10 wherein the fullerene is C₇₀.

13. The cosmetic composition according to claim 1 wherein the fullerene is a mixture of C₆₀ and C₇₀.

14. The cosmetic composition according to claim 1 wherein the fullerene clusters are formed by fragmenting fullerites to nanometer size in a liquid media.

15. The cosmetic composition according to claim 14 wherein fragmentation is effected through shock wave, electrohydraulic impact, ultrasonic wave treatment or acoustic wave treatment.

16. The cosmetic composition according to claim 14 wherein fragmentation is effected by electrohydraulic impact.

17. The cosmetic composition according to claim 14 wherein the liquid media is water, alcohol, synthetic oil, natural oil or mineral oil.

18. The cosmetic composition according to claim 1 wherein the carrier is water, ethanol or mixture thereof.

19. The cosmetic composition according to claim 7 wherein the carrier is water, ethanol or mixture thereof.

20. The cosmetic composition according to claim 19 which additionally contains emollients, and anti-oxidants and emulsifiers.

21. The cosmetic composition according to claim 20 wherein the carrier is water.

22. The cosmetic composition according to claim 1 in the form of a cream.

23. The cosmetic composition according to claim 7 in the form of a cream.

24. The cosmetic composition according to claim 21 wherein glycerol is additionally present.

25. The cosmetic composition according to claim 24 which additionally contains petrolatum, mineral oil, glycerin, isopropyl palmitate, PPG-2-myristyl ether propionate isopropyl palmitate and lanolin alcohol.

26. The cosmetic composition according to claim 7 additionally comprising white petrolatum in an amount ranging from about 0.5% to about 1.5% by weight, Lanolin Alcohol, ranging from about 0.5% to about 1.5% by weight, PPE-2-myristyl ether propionate ranging from about 3% to about 6% by weight mineral oil ranging from about 5% to about 10% by weight, triethanolamine ranging from about 0.2% to about 0.8%, glycerol ranging from about 2% to about 4% by weight, stearic acid ranging from about 15% to about 20% by weight, isopropyl palmitate ranging from about 10% to about 15% by weight of the total cosmetic composition and the remainder is water.

27. The cosmetic composition according to claim 26 additionally containing a fragrance and an anti-oxidant.

28. A method of reducing damage by free radicals to the healthy skin or mucous membranes of a mammal comprising applying to the healthy skin or mucous membrane of said mammal the cosmetic composition according to claim 1 in an amount effective to protect the skin form free radicals.

29. A method of deactivating skin toxins present on the skin of a mammal which comprises applying to the skin of said mammal the cosmetic composition according to claim 1 in an amount effective to deactivate the skin toxins.

30. A method for the treatment of wounds in a mammal which comprises administering onto the wound a wound healing effective amount of the cosmetic composition of claim 1.

31. The method according to claim 30 wherein the wound is selected from the group consisting of aseptic wounds, contused wounds, incised wounds, lacerated wounds, non-penetrating wounds, open wounds, penetrating wounds, perforating wounds, puncture wounds, septic wounds, infarctions and subcutaneous wounds.

32. The method according to claim 30 wherein the wound is selected from the group consisting of ischemia, ulcers, pressure sores, fistulae, and thermal burns.

33. A method for the treatment or prophylaxis of infection on the skin of a mammal comprising administering to the surface of the skin of a mammal, a pharmaceutically effective amount of the composition according to claim 1.

34. The method according to claim 33 wherein the infection is caused by a microorganism.

35. The method according to claim 34 wherein the infection is caused by bacteria.

36. A method for the treatment of an inflammatory condition in a mammal which comprises administering topically to the skin of the mammal a pharmaceutically effective amount of the composition according to claim 1.

37. The cosmetic composition according to claim 1 wherein the fullerne clusters are associated with a terpene or derivative thereof, said derivative consisting of an alcohol, aldehyde, ketone, lower alkyl ether or lower alkyl ester of the terpene.

38. The cosmetic composition according to claim 37 wherein the terpene contains 10 m carbon atoms, where m is 1 to 10.

39. The cosmetic composition according to claim 38 wherein the terpene or derivative thereof is geraniol, citrinellol, citrel, linalyl acetate, menthol or terpeneol.
